SEQUENCE LISTING (19 sequences)

SEQ ID NO: 1 - DNA, 14107 bp, synthetic construct
  source 1..14107: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 2 - DNA, 11571 bp, synthetic construct
  source 1..11571: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 3 - DNA, 17068 bp, synthetic construct
  source 1..17068: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 4 - DNA, 10995 bp, synthetic construct
  source 1..10995: mol_type = other DNA
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgccagtgccaagctaattcgaggggatttaaattggtcgatctggttgtactgaggacttctacaagaaggacagctctgtttgaggtcggattgggatcgcttgaaaagcttgttctgttacgtttgatccctccaccatgagcaacgagctgaattgaaactcagaaaggtcttgctgcatcaactgatgcaggtggcaaagacactgaagggtcgatgaggtgcagagcgaatcgacagcagctgtaagggttcctcttccttctcctcctcctcctcctcttccttcgcagcaccgcattatgcttgaattgtgttgcgctccggcgtcactcccatgaacatgaaaacacggaccagcaccgaacctacgttttcatcgacgatttagtgttgaaacctcacctataatgacttcaatggctttgcaagtgcattagtagtgattacatacaataattttaaatctcaattggcactaaataatgtcaacaagattaaattactaccaaatccagttgggttacaaaatgaaacaaaagcatctgacaatctcaaacatgaaaagactcgtgcacatggtctcaaaaaagttagaaggtaatgattgagcaagaaaatactttatcacattcattgatgattgagtggaatctgaagggagatagcataaaggtcttacataacatttattgataattgagtacaattgaagtaaagcctggacaacaacttcaatacatctccaccgcggtggcggccgctctagaactagtggatcccccgggctgcaggaattagcttatcgataccgtcgacctcgagggggggcccggtaccaagcttgctagcgaattcgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgaattcgatatcccgggcgcgcggtaccataacttcgtataatgtatgctatacgaagttatcctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcatggagaaccgacctgcaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcagggactcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggacggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccataacttcgtataatgtatgctatacgaagttatgcatgcgatagcagctgtccttctcaagtggaaaggtagtgtgaataggtggagtgagggaagagcttctcctcaagtgctctgacaactttagggtttatttgcaggaagctaccaagtgttgaatgaccaagccatcgctttatgattgcaagagtactccctgacacgcgtggaacaggggattaagtgaatgggagggaatgcgtttctgagaggaatctcatgttctactatgagaatgtagagaaaacaagaggcgtccgtgttgcacttgtgtgggtgcatgtcgtacttgcaagttttgtagaggaaaacttaagttacttatgtttcgcaagagttttaggtttcgagggatcgccactgccagatttagacacacacttttcaaagagtgagagggggcgaggttgaggggagaacgctggcgcgcacagggggcgcctgatggctgatcggcctcaagcacatgtaacttttcatgaaattcgtctggaagtgaaagagccgaagtacaaaagcgtgagtatcatcttaagcggagctggagataagggagggggcgaaagcggggaagaatgctgagtgtagtgcgggtgcacctcccgaccgacattcccattgtaggctgcgagttgtcggcctatgtgctgttgcgacggggagattcgagtctttctcctgaagatgttaccgaggcatcgtctacggatggctattttctccagtgtcgatggtatctgtccttacttctaaacttttctttttgtttattgtggaacattcatgctaacgttcttcttgaaagacgacagtccgtgagttgaatttaaatcccatctcgagatctagcttggc

SEQ ID NO: 5 - DNA, 5340 bp, synthetic construct
  source 1..5340: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagccaaaatgcatggccgcgctccctgctgcgagaaggtgggcatcaagcgcggccgctggaccgcggaggaggaccagatcctgagcaactacatccagtcgaacggcgagggctcctggcgcagcctgccaaagaacgccggcctgaagcgctgcggcaagtcgtgccgcctgcgctggatcaactacctgcgctccgacctgaagcgcggcaacattacccccgaggaggaggagctggtggtcaagctgcacagcaccctgggcaaccgctggagcctgatcgccggccacctgcccggccgcaccgacaacgagatcaagaactactggaacagccacctgtcgcgcaagctgcacaacttcatccgcaagccctccatcagccaggacgtgagcgctgtgatcatgaccaacgcctcgagcgcgcctcccccgccccaggccaagcgccgcctgggccgcaccggccgctcggccatgaagcccaagatccaccgcaccaagacccgcaagaccaagaagaccagcgcgccgccggagcccaacgccgacgtggccggcgccgacaaggaggcgctgatggtggagagctcgggcgctgaggcggagctgggccgcccctgcgactactacggcgacgactgcaacaagaacctgatgagcatcaacggcgacaacggcgtgctgacgttcgacgacgacatcatcgacctgctgctggacgagagcgaccccggccacctgtacacgaacaccacctgcggcggcgacggcgagctgcacaacatccgcgacagcgagggcgcccgcggcttcagcgacacgtggaaccagggcaacctggactgcctgctgcagagctgcccctccgtggagagcttcctgaactacgaccaccaggtgaacgacgctagcacggacgagttcatcgactgggactgcgtgtggcaggagggcagcgacaacaacctgtggcacgagaaggagaaccccgacagcatggtgagctggctgctggacggcgacgacgaggcaacgatcggcaacagcaactgcgagaacttcggcgagcccctggaccacgacgacgagagcgccctggtggcctggctgctgagctaaccggtgcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcagaagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagagatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 6 - DNA, 8635 bp, synthetic construct
  source 1..8635: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggatcccacacacctgcccgtctgcctgacaggaagtgaacgcatgtcgagggaggcctcaccaatcgtcacacgagccctcgtcagaaacacgtctccgccacgctctccctctcacggccgaccccgcagcccttttgccctttcctaggccaccgacaggacccaggcgctctcagcatgcctcaacaacccgtactcgtgccagcggtgcccttgtgctggtgatcgcttggaagcgcatgcgaagacgaaggggcggagcaggcggcctggctgttcgaagggctcgccgccagttcgggtgcctttctccacgcgcgcctccacacctaccgatgcgtgaaggcaggcaaatgctcatgtttgcccgaactcggagtccttaaaaagccgcttcttgtcgtcgttccgagacatgttagcagatcgcagtgccacctttcctgacgcgctcggccccatattcggacgcaattgtcatttgtagcacaattggagcaaatctggcgaggcagtaggcttttaagttgcaaggcgagagagcaaagtgggacgcggcgtgattattggtatttacgcgacggcccggcgcgttagcggcccttcccccaggccagggacgattatgtatcaatattgttgcgttcgggcactcgtgcgagggctcctgcgggctggggagggggatctgggaattggaggtacgaccgagatggcttgctcggggggaggtttcctcgccgagcaagccagggttaggtgttgcgctcttgactcgttgtgcattctaggaccccactgctactcacaacaagccaaaatgcatgccaccggcatccagaaccagaagatcgtgcccgagaacctgcgcaagcagctggccattgcggtgcgctctatccagtggtcgtacgcgatcttctggagcaacagcgtggcgcagccgggagtgctggagtggggcgacggcttctacaacggcgacatcaagacgcgcaagaccgtgcagagcgtggagctgaaccaggaccagctgggcctgcagcgcagcgaccagctgcgcgagctgtacgagagcctgagcctgggcgagaccaacacgcaggcgaagcgccccaccgcggccctgagccccgaggacctgaccgacgccgagtggttcttcctggtgtgcatgagcttcatcttcaacatcggccagggcctgccgggccgcaccctggcccgcaaccaggctgtgtggctgtgcaacgcccaccgcgccgacaccaaggtgttctcccgcagcctgctggccaagtccgcgtccatccagaccgtggtgtgcttcccctacagcgagggcgtggtcgagctgggcgccacggagctggtgcccgaggacctgaacctgatccagcacatcaagacctcgttcctggacagccccgccaccgtgcccaagatcccgaactacgtgagcaacagcatcaccaataacaatgacctgatctgcgaggcgctggagcacgccaacatccccgagaacgacctggaccagctgctgaactgccccgacaccaacatctgctcccccgacaactctctggacgacttcgccgacaacctgctgatcgacgagagcaacctggcggagggcatcaacggggaggtgccccagacgcagagctggcccttcatggacgacgccatcagcaactgtctgaacagcagcatgaacagcagcgactgcatcagccagacccacgagaacctggagagcttcgcgccgctgagcgacggcaagggcccccccgagacgaacaactgcatgcacagcacccagaagtgcaaccagcagatcgagaacaccggcgtgcagggcgacgaggtgcactaccagggcgtgctgagcaacctgctgaagtcgtcccaccagctggtgctgggcccgtacttccgcaacggcaaccgcgagagctcgttcgtgagctggaacaaggacggcagctcgggcacccacgtgccgcgctcgggcacctcccagcgcttcctgaagaaggtgctgttcgaggtggcccgcatgcacgagaactcgcgcctggacgcgggcaagcagaagggcaacagcgactgcctcgccaagcccaccgctgacgagatcgaccgcaaccacgtgctgtccgagcgcaagcgccgcgagaagatcaacgagcgcttcatgatcctggcgagcctggtccccagcggcggcaaggtggacaaggtgagcatcctggaccacaccattgactacctgcgcggcctggagcgcaaggtggacgagctggagagcaacaagatggtgaagggccgcggccgcgagagcaccaccaagaccaagctgcacgacgccatcgagcgcacgagcgacaactacggcgcgacccgcacctccaacgtgaagaagcccctgaccaacaagcgcaaggcctccgacaccgacaagatcggcgcggtgaacagccgcggccgcctgaaggactcgctgacggacaacatcaccgtgaacatcaccaacaaggacgtgctgatcgtggtgacatgcagcagcaaggagttcgtcctgctggaggtgatggaggcggtgcgccgcctgtccctggacagcgagaccgtgcagagctccaaccgggacggcatgatcagcatcaccatcaaggctaagtgcaagggcctgaaggtggcctccgccagcgtgatcaagcaggcgctgcagaaggtgaccatgaagagcctgctggcgatccaccccaccgaggcgcgccacaagcagaagattgtggcccccgtgaagcagaccctgaacttcgacctgctgaagctggcgggcgacgtggagtccaacccgggccccatggagaagaactgccgcggcgtgcgcaagggcacctggaccaaggaggaggacaccctgctgcgccagtgcatcgaggagtacggcgagggcaagtggcaccaggtgccccaccgcgccggcctgaaccgctgccgcaagtcctgccgcctgcgctggctgaactacctgcgcccgaacatcaagcgcggccgcttcagccgcgacgaggtggacctgatcgtgcgcctgcacaagctgctgggcaacaagtggagcctgatcgccggccgcatccccggccgcaccgcgaacgacgtgaagaacttctggaacacccacgtgggcaagaacctgggcgaggacggcgagcgctgccgcaagaacgtgatgaacaccaagaccattaagctgaccaacatcgtgcgcccgcgcgcgcgcaccttcaccggcctgcacgtgacctggccccgcgaggtgggcaagacggacgagttctccaacgtgcgcctgaccacggacgagatccccgactgcgagaagcagacgcagttctacaacgacgtggcgtccccgcaggacgaggtggaggactgcatccagtggtggtcgaagctgctggagaccaccgaggacggcgagctgggcaacctgttcgaggaggcgcagcagatcggcaactaaccggtgcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcagaagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagagtcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgacacaagaatccctgttacttctcgaccgtattgattcggatgattcctacgcgagcctgcggaacgaccaggaattctgggaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgataaggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 7 - DNA, 11118 bp, synthetic construct
  source 1..11118: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 8 - DNA, 11201 bp, synthetic construct
  source 1..11201: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggccctgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggatgaatcaagtattattccggcagagaaagtggccggagctgagaaaaaagagcttcaagggctgcttaagacggcggttcaatctgtggactggacttatagtgtcttctggcaattttgtcctcaacaacgggtcttggtgtgggggaatggatactacaacggtgcaataaagacgaggaagacaactcaaccagcggaggtgacggcggaagaggctgcgttagagaggagccaacagctcagggagctttatgagacacttttagccggagagtcaacgtcagaagcaagagcatgcaccgcattgtcaccggaggatttgacggagacagaatggttttatctaatgtgtgtgtctttctcttttcctcctccatctgggatgccaggaaaagcgtatgcaaggaggaagcacgtatggctaagtggtgcaaatgaagttgacagtaaaactttttctagagctattctcgctaagagtgctaaaattcagacagtggtttgcattccaatgcttgatggtgttgtggaactaggcacaacgaaaaaggtaagagaagatgtagagtttgttgagctcacaaagagtttcttctatgaccactgcaagacgaacccaaagccggctctttctgaacactccacctacgaagtgcatgaagaagccgaagacgaagaagaagtagaagaagagatgacaatgtcagaggaaatgaggcttggctctcctgatgatgaagatgtttccaatcaaaatctacactctgatcttcatattgaatcaacccatacgttagacacacatatggacatgatgaatctaatggaggaaggtggaaactattctcagacagtaacaacacttctcatgtcacaccccacaagtcttctttcagattcagtttccacatattcttacatccaatcatcgtttgccacgtggagggttgagaatggcaaagagcatcagcaagtgaaaacggcgccgtcgtcacaatgggtgctcaaacaaatgatcttcagagttcctttcctccatgacaacactaaagataagaggctaccgcgggaagatctgagccacgtagtagcagagcgacgcaggagggagaagctgaacgagaaattcataacgttgagatcaatggttccatttgtgaccaagatggataaagtctcaatccttggagacaccattgcgtacgtaaatcatcttcgaaagagggtccatgagcttgagaatactcatcatgagcaacagcataagcggacgcgtacttgtaagagaaaaacatcggaggaggtggaggtttccatcatagagaatgatgttttgttagagatgagatgtgagtaccgagatggtttgttgcttgacattcttcaggttcttcatgagcttggtatagagactacggcagttcatacctcggtgaacgaccatgatttcgaggcggagataagggcgaaagtaagagggaagaaagcaagcatcgctgaggtcaaaagagccatccaccaagtcataatacatgatactaatctatagcccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 9 - DNA, 2214 bp, Catharanthus roseus
  source 1..2214: mol_type = genomic DNA
atggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagttcgagcagctgctgctcatgggcatgctcataatgttgatgaaaattaccaagtcctagtgaagaagcagcctttgaatttttcaggagataagccttttactcctattttggataccatcaactaccctacccatatgaagaatctctctcttcaggagcttcaacaattagctgacgaattaagggaggagatagtgtattctgtggccaaaacgggtggtcacttaagttccagcctaggtgtggccgagctcactgtggcactccaccacgtgttcgaaaccccaaatgataaaatcatatgggatgttggccaccagacatatgggcataagattctgacagggagaaggtcaaagatgcataccatgagacaaacttgcggacttgcaggtttccccaaaagagatgaaagtgcccatgatgcatttggagttggccatagttccaccagcatttctgccggtttaggaatggcaataggacgagatttattaggtaagaaaaatcatgtaataacggtaataggagatggagctatgacagccggacaagcatatgaagccatgaataatgctggatatcttgattctaatctcataatcatattgaatgataacaaacaagtttctctcccgactgccacaattgatggtcctgcacctcctactggtgctctaagcaatgccttaactaggcttcaatctagtagaaaatttcgactacttcgggaggctgcaaaaggtgtgacaaagcaattagggggacaagttcatgaaatggcagctaaagtggatacatatatgagagggatgaatagccaaagggcttccttatttgaagatttaggaatgtattatattggtccacttgatggtcacaatatggaagatcttgttgatattttagagaaagtaaaatctgttcctgcaacgggccctgttcttattcatattattactgagaaaggaaaaggatatcctcctgctgaagctgccgccgacaaaatgcatggggtggtgaaatttgagccaatgacaggaaaacaattcaagtcaaggtcaaaaacaaagtcatatacaaattattttgctgagagtttaataagtgaagctagggaagatgataagattattgccattcatgctgctatgggaggaggaacaggactcaatttattccagaaacactttcctgaacgatgctttgatgttgggatagctgagcaacatgctgtcacttttgctgctggcttagcttctgaaggtctcaagcctttttgtgctatttactcatctttcttacaaagaggttatgatcaggtggttcatgatgtggaccttcaaaaaattccagtaaggtttgctatggatagagcagggctggtgggtgcagatggacccactcattgcggtacatttgacacaacttttatggcttgtttgcctaatatggtggtcatggctccttcagatgaaaccgaattgatgcacatggttgccacagcagctgccattgatgataggcctagttgctttcgatatcccagaggaaatggcattggtgcaattcttccaccaaacaacaaaggagctccattacagattggtaaaggaagaatattgttggagggaacaagagttgccattcttgggtatggagcaatggtgcaaaactgtttaatggctgctcagcttcttgaaattaatggaatatcagctacagttgttgatgcccgattttgcaaacctcttgatggagatttgattagaaaattggctcaacaacatgaagtccttatcactgttgaagaaggatccattggtggttttggttcccatgtggcacagttcttggctttgaatggattgcttgatggaaatttaaagtggagggcaatgatgcttcctgacagatatattgatcatggagctcaaaatgaccaaattgaagaggctggcctaactccaaagcatattgctggcactgtgttttctctactcagcaaaaacaagaatactcttcaagttctccagttttag

SEQ ID NO: 10 - DNA, 1152 bp, Picea abies
  source 1..1152: mol_type = genomic DNA
atggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagttcgagcagctgctgctcatgggcatgctcattgctcaaacacaaatgcccagttggccaatttgtctgaacaagtaaaggggaaggtcactgaatttgatttcaaggagtacatgcgttccaaggcaatgtcagtgaatgaggcactggacagggctgtcccacttcgttatcctgaaaaaatacatgaagctatgaggtattctcttctagcaggagggaagcgagttcgtcctattctgtgcattgcagcatgtgagcttgtgggagggagcgaggagctcgccatgccaactgcctgtgcaatggaaattatccacacaatgtctttgattcatgatgacttgcctcccatggacaatgatgatttacgccgaggtaagcctacaaaccacaaggtcttcggtgaaggcactgctgttcttgcgggggatgctcttctttcatttgcatttgagcacattgcagtgtccacaagcaagactgtggagtctgatagggttttgagggtggtatctgaattgggtagagcaataggctcggaaggggttgcaggtgggcaggttgctgatattaccagtcaggggaatccttctgttggccttgagactctggaatggattcacattcacaagactgcagtgctcttggagtgctccgttgctagtggggcgatcatcggtggtgcttctgatgatgagattgagagagttcgaaagtatgcccgttgcgtggggcttctgtttcaggttgtggatgacattctcgatgtcacgaaatcatcagaagaattgggtaagactgcagcaaaggatttgcttagtgataaggccacttatcccaagctcatgggtttggagaaagcaaaggagtttgctgatgaattgttgggcaaagctaaggaggagttatctttcttcaacccaactaaggctgcacctctgttgggtcttgcagattacattgcacaaagacagaactga

SEQ ID NO: 11 - DNA, 1854 bp, Catharanthus roseus
  source 1..1854: mol_type = genomic DNA
atggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagttcgagcagctgctgctcatgggcatgctcattctatgccatcatcttcatcatcatcatcatcttcatccatgtctctgcctttggcaactccattgatcaaagacaatgaatctctcatcaagttcttgcgccaacccctggtgcttcctcatgaggttgatgacagcacaaaaaggagggaattgttggaaagaacaagaaaagaactagaattaaatgcagaaaaaccattggaggccttgaagatgatagatataattcaaagattgggattatcatatcattttgaagatgatattaattcaattctcacaggattttcaaatattagcagccaaactcatgaagatctcctcactgcttcactttgttttcgattgcttcgacacaatgggcataagatcaatcctgatatattccaaaaattcatggacaacaatggaaagtttaaagattcattaaaggatgacacattaggcatgttaagcttatatgaagcttcatatttgggagccaatggagaagaaatattgatggaagcccaagaattcaccaaaactcacctgaaaaactcattgccagccatggcaccatctctttctaagaaggtttctcaagctttagagcaaccaagacatagaagaatgttgaggttagaagctagaagatttattgaagaatatggtgctgaaaatgaccataatccagaccttcttgagcttgcaaaattggattataacaaagtccaatctctacaccaaatggaattgtctgagataacaaggtggtggaaacaattagggcttgtggataaactcacctttgctcgagatcgaccccttgaatgctttctttggacagtgggattattaccagagcctaagtattcaggttgcagaattgagcttgcaaaaaccatagccattttgcttgtcattgatgatatctttgatactcatggtaccctagatgagcttcttctattcactaatgccattaaaagatgggatcttgaggccatggaagatttaccagaatatatgagaatttgttacatggcattgtacaatactactaatgaaatttgctataaagttcttaaggaaaatggttggagtgttcttccttacctaaaggcaacgtggattgatatgattgaaggattcatggttgaagcagaatggttcaattctgattatgtaccaaacatggaagaatatgtagaaaatggagttagaacagcaggatcatatatggccttagtccatttgttctttctaatagggcaaggtgtcactgaagataatgtgaaattactgattaaaccctatccaaagctcttttcctcctcaggaagaatccttcgcctttgggatgatttgggaactgcaaaggaggaacaagaaagaggagacttggcatcaagcatacaattgtttatgagagagaaagagataaaatcagaagaagaaggaaggaaaggaatattggaaattatagagaatttatggaaagaattgaatggagaattagtttatagagaagaaatgcctcttgcaataatcaagacagcattcaacatggcaagagcttcccaagttgtgtatcaacatgaagaagacacctatttttcaagtgtagataattatgtaaaagctttgttttttacaccttgtttttaa

SEQ ID NO: 12 - DNA, 1482 bp, Catharanthus roseus
  source 1..1482: mol_type = genomic DNA
atggattaccttaccataatattaactttactatttgccttgactctctatgaagccttcagctacctatccagaagaaccaaaaaccttcctccaggaccatcgccattgccgttcatcggaagcctccatttattaggcgaccaaccacacaaatccttagcaaaactttccaaaaaacacggtccaattatgagtctcaaattaggccagatcactacaatcgtcatatcttcatcaacaatggcgaaagaagttcttcaaaaacaggatttagcattttcaagcagatcagttccaaacgcactccacgctcacaatcaattcaaattctccgttgtatggcttccggtagcctcacgatggagaagtcttcgaaaagttttgaattctaatatattttccggcaatcggctcgacgctaatcaacatttgagaactagaaaagtacaggaactaattgcgtattgccggaaaaatagccagagcggagaagcggttgacgtcggccgagctgcttttagaacttcgttgaatttgttgtcgaatttgattttttcaaaggatttgacggatccttattcggattctgccaaggaattcaaggatttggtttggaatataatggttgaggcggggaaacctaatttggtcgatttttttcccctgcttgaaaaagttgatcctcaaggtatacgacatcgtatgacgattcactttggggaagttcttaagctttttggtggacttgttaatgaaagattggagcaaagaagatcaaaaggggaaaaaaatgatgtgttggatgtacttctaactaccagccaagaaagccctgaggaaatcgatagaactcacattgagcgaatgtgcttggacctgtttgtagcagggacggacacaacatcaagcacattagaatgggcaatgtcagaaatgcttaaaaacccagacaaaatgaagaaaacccaagatgaacttgcacaagtaatcggcagaggaaaaacaatagaagaatccgatattaaccgcttaccttacttaagatgcgttatgaaagaaaccttaaggatacatccaccagttcccttcttaattcctcgcaaagtggaacaaagtgttgaggtttgtggatacaatgtccctaaaggatcacaagttcttgtgaatgcttgggcaattggacgtgatgaaactgtttgggatgatgctttggcattcaaacccgagagatttatggaatctgaattggatatccgtggaagagatttcgagctgattccgttcggtgctggccgaagaatttgcccagggttgccattggcactaaggactgtgcctttgatgcttggttctttgttgaactcttttaattggaagcttgaaggtgggatggctccaaaagatttggatatggaggagaagtttggtattacactgcagaaggctcatcctttgcgtgctgtaccaagcaccctttaa

SEQ ID NO: 13 - DNA, 1137 bp, Catharanthus roseus
  source 1..1137: mol_type = genomic DNA
atgaccaagaccaattcccctgctccttctgtcatcacttgcaaagctgctgtggtatggaaatcaggggagccaccaaaggtggaagagatacaagttgatccaccaaaggcctcagaagttaggattaagatgctttgtgccagtttgtgccacactgatttccttgcctgcaatggccttcctgttccattgttccctcgcattcctggacacgaaggagtcggaatgatcgagagcgttggagaaaatgttacgaacctaaaagaaggagacatagtgatgccactttacttgggagaatgtggggaatgcttgaattgcaaatcaggaaggacaaatttgtgccacaaatatcctttaggttttagtggattattgcttgatggaacatcaagaatgtcaattggagagcaaaaagtatatcaccatttcagctgttcgacatggtcagagtatatagtgattgaagcagcttatgcagtgaaggtagatccaagggtttctcttccacatgctagctttctttgctgtggattcaccactggttttggtgctacttggagagatgtcaatgttgtcaagggctctactgtggctgttctaggccttggtgctgttggacttggggctgtgcaaggagctaaatcacaaggagcatcaagaattataggtttggatatcaacgacaagaaacgtgaaaagggagaagcatttggaatgactgaatttataaatccaaaaggttcaaacaaatcaatttctgaactaataaatgaagcaactggtggattaggacttgactatgtctacgaatgcactggagttccagctttactcaatgaagccattgaatcctctaaagtgggacttggaactgcagtattgattggggcaggacttgaaacaagtggagaaatcaaattcattcctcttctgtgtggtagaactgttaaaggttcaatttatggtggagtaagacctaaatcagacctccccactttaattgaaaagtgcataaacaaggaaattccaatggatgaactaatgactcatgaagtttcattgtctgagataaacaaaggatttgagtaccttaagcatcctgattgtgtcaaagttgttatcaagttctaa

SEQ ID NO: 14 - DNA, 1167 bp, Catharanthus roseus
  source 1..1167: mol_type = genomic DNA
atgagttggtggtggaagaggtccattggtgctggaaagaacttaccaaaccagaacaaagaaaatggagtttgtaaaagctacaagagtgtggcactagtagtaggagtcaccggcattgttggcagtagtttagccgaagttctaaagctaccagacacccctggaggtccctggaaggtatacggcgtggcaaggcgcccatgccctgtatggctagccaaaaaaccagttgagtacatccaatgtgatgtctcagataatcaagaaacaatttctaaattatctccacttaaggacataactcatatattctatgtttcttggattggatcagaagattgccaaacaaatgctacaatgttcaaaaacatccttaattcagtgattcctaatgcttcaaatctccaacatgtgtgtctccaaacaggaatcaaacattattttggtatttttgaagaaggttctaaagttgtaccacatgattcgccttttaccgaggatttacctaggctaaatgtcccaaatttctaccatgatcttgaagatatactgtatgaagaaactggcaagaacaatttaacatggtctgtccatagaccagctctggttttcgggttttccccatgtagtatgatgaacattgtcagtacattatgcgtttacgccacaatttgtaaacatgaaaataaggccttggtttatccaggtagtaaaaattcatggaattgttatgctgatgctgttgatgcagatttagtagccgagcacgaaatttgggcagcagttgatcctaaggctaaaaatcaagtattgaattgtaacaatggggatgtttttaaatggaaacatatttggaagaaattagcagaggaatttgggattgaaatggtgggatatgttgaaggtaaagaacaagttagtttggctgagttaatgaaggataaagatcaagtttgggatgaaattgttaagaagaacaatttagtacctactaaattgaaagaaattgctgccttttggtttgctgatattgctttttgttctgagaatttgattagtagtatgaacaaaagtaaggaattagggttcttgggttttaggaattcgatgaaatcgtttgtttcttgtattgataagatgagggattataggtttattccttag

SEQ ID NO: 15 - DNA, 483 bp, Nepeta mussinii
  source 1..483: mol_type = genomic DNA
atggcttcgaagcttgaaatagaaattgagttgaaaagtgatgtagaaaagatgtggaaacacttcaaagaattcaccaaattattccccaaggcttgtcctcatctttacgagaagattgatgtgatcgagggagatgggatatcagttggaacaatctttgtcagtaccctgaaaccaacagagttgaaccccgtggttatggtgacgaaggagaagattgattttttagatgatgagaacaaaatgttgaggtatagttacatggaaggggagatattaaagaattacaagaatttgaggggcacagtgcatatgagcagcagcaaaagtggtggaactatattcaaatattcaggagaattcgaaaaggcaaatgaacaagtccctgatcctgttttcttcaaagattttatggtcatagttttccaaggtttggatgattatattctcaagggcatgaaccatacatgtcaaaattag

SEQ ID NO: 16 - DNA, 1548 bp, Catharanthus roseus
  source 1..1548: mol_type = genomic DNA
atggcgaccatcactttcgattcactaaacccggtgaccgtcgccatctcagccggttttcttctcctccttatcatctttgtgaaatcaagaactggttcatccaaaagaaaaccgccgggtcctccaggatggccgattttcggcaatatgttcgacctcggagatttacctcaccaaacgctgtacaagctcaagtccaaatacgggccgatcgtatggctccagctcggctcaattaatactatggttgttcaaaatgcagtttccgccgccgagctttttaagaaacacgatgtccccttttgtgaccgaaaagtccctgatactctcactgccttcaatttcaaccagggctccctgggtatgaatacttacggcggacattggcgagtgcttagacgcctctgttccatggagtttttggtaaataaacgtatgaacgaaactacagatctaagaaggagaattgaggacaatatggtccgttggattgaagaagattcattggcatctaaagcacaaggaggaaccggagcggttcaattgagcaggtttctttttctaatggcgtttaatttggtgggtaaccttatgttatcgagagatttaatggataataaggatccagaaggaagggaattttttgattgtatgaatgagatattggaattggctggtactcctaatattgctgattttttgcctttgttgaagaaattggatccacttggaatgaagaaaaggatggttgataatatgtctagaactatgaaaatttcttcaaagtttgttcaagaaagacttgataatagaaaagctggaaagattaatgaaaagaaagatttcttggatgttatgcttgaataccaaggtgatggtaaagatggtcccgataaattcactgagcaacatgtcaacattgtcatcatggaaatgttttttgcaggatcagagacaacaagtatcagcattgaatggggattcacagaacttctccgaaacccacacgcattcaagaaagtaagggaagagatcgacagagtagtgggagtaaacagaatggtcgaagagaccgacatggagaatttgccgtatttgcaagcagttgtaaaggagacgcttagattgcatccggcacttccaatgcttctaccaagaaacacaatggaagacactgaatacatgggatacctcattcctaaaggcacacaagttttcgtgaatgcatgggcaatcggaagggatcctgagtactggcaggaccctttgagtttcaaaccagaaaggtttatcaattccagtgttgaatataaaggacaacattttgaattgattccttttggttctggaagaagaatttgtgtgggatttcctttggctcatagagttgttcatctcactttggctacattagtccaggcttttgattgggatcttggtgctggcgttaaaccacaagatattgatttggaagagagattgggattgactctgaggaagaagaatccattgaatgtcatccctaagaagagagttcatatctaa

SEQ ID NO: 17 - DNA, 1449 bp, Catharanthus roseus
  source 1..1449: mol_type = genomic DNA
atgggttctcaagaaacaaatttgcctcctcatgttctcatattccctttgccaattcaaggccatgtgaactccatgctcagactagcagaacttctctgtctagctgaattagatatcacttttattgtttcagagttcagccacagtcgcttaattaagcatacaaatgttgcctcccgttttgcccgttacccaggatttcagttccagcccatatccgatggccttcccgatgatcaccccagagcaggcgaaagagtcatggatattttgccttcaactaaaaatgtaacgggacccctgtttaagcagatgatggttgagaataagtgcttttcttctgctactcgaaggccgattacttgcataatcgctgatggggttttgagctttgctggggatttcgctcaagaaaaaggaatccctcttatttacttcagaactgtttctgcttgttctttctgggcttgtttttgtatgcctgaactaattgaatctggtgacattccgattaaaggaaatggtatggatctgatagtgaaaagcgtgccggggatggaaacttttctcaggcgacgagatctgccggggttttgccgagtcaatgacattaatgaaccgaaactccagatcctgaaaacagagacaagacagacaacgagagcccaagcggctattttgaacacttttgaggatttggaaggaccaattctatctcaaattcgcaaacacatgccaagacttttcacaattggaccaagtcattcccacctaacatctagacttgaaaccaagaatatcaagactttaatttcctccggtagtttctgggaagaagatcgaagctgtgtggattggcttgatgcacagccaccaagatctgttttatatgtaagttttggaagtattacagttgtaacaagagaccaactcctggaattctggtatggtcttgttaatagtggacaacggttcttgtgggtaatgaggccagattcaattatgggcaaagatgggcaaagccagattcctgcagatcttgaagagggcacaaaagccagaggttatatggtgggatgggcacctcaagaagaggtcttgaatcaccctgccattggaggatttctgactcacagtgggtggaattcaactttagagagtattgtggctggtgtcccaatgatatgttggccatactttgcggaccaaatgataaacagcagatttgtgagtgaaatttggaagattggattggatatgaaagatacatgtgatagagagacaattgtgaagatggttagggagttgatggagattaggaaagatgagttcttgcaaagggctgatcatatggctaaattggcaaaagaggctgttagtgaaggcggatcctcatattctaacttggatggcctcgttgattacataaaatcactgattatttga

SEQ ID NO: 18 - DNA, 1566 bp, Catharanthus roseus
  source 1..1566: mol_type = genomic DNA
atggaattgaacttcaaatcaattattttcttggtttttgttagtctaaccctttactgggtctatagaatcttggattgggtatggtttaagcccaagaagctggaaaaatgcttaagagaacaagggtttaagggaaatccttacagattgttcttgggagatcagtatgatagtggaaaattgatcagacaagccttgactaaacctattggtgttgaagaagatgttaagaaacgaattgtacctcatatactcaaaactgttgggactcatggcaagaagtcttttatgtgggttggaagaataccaagggtgaatatcacagatccagagctgattaaagaggtcttaacaaaatactataagttccagaagaatcatcatgatcttgacccaattaccaaattgcttttgactggaattggaagcttagaaggtgacccttgggctaaacgccgaaaaatcatcaatgctgcttttcactttgaaaagttaaagcttatgcttcctgcattctatttgagttgccgtgatatggtgacaaaatgggataataaggtccctgaaggaggatcagcagaggtggatgtgtggcatgatattgaaaccttaactggagatgtaatttcaagaacattatttggaagtaacttcgaggaagggagaagaatatttgaactcatgaaagaacttactgctcttactattgatgtcattcgctcggtttatatccctggacagaggtttcttccaactaagaggaacaataggatgagagcaattgacaaagaagttagagtaagaataacagaaattatcaataagaaaatgaaggtaatgaaatcaggagaagcagcaagtgctgctgatgattttttgggaatattgttggaatgcaatctgaatgaaataaaggagcaaggaaataacaaaagtgctggaatgactattggagagattattggtgagtgcaaactcttctattttgctggccaggacactacctcgactttactcgtgtggacaatggtcctcttgtctagattccccgaatggcaaactcgtgccagagaagaagtttttcaagtctttggtaataaaaccccggactatgatggtatcagccatcttaaagttataacgatgatcttgtacgaggttttgaggttatatactccagtggctgaactgacaaaagtggctcatgaagctacacaactgggaaaatacttcattccagcgggcgtgcagctaatgatgccacaaatcctacttcatcatgaccctgaaatatggggtgaagatgtaatggagttcaaaccagaaagatttgcagagggagttctcaaggcaactaagagccaaggctctttttttccatttagcttgggaccaagaatgtgcattggccaaaactttgctctattggaagccaaaatggcgatgtctctaatcttgagacgtttttctttcgagctttctccctcctatgttcatgctcctttcactctcatcacaatgcaaccccaatatggtgctcatttgattctgcacaaactctaa

SEQ ID NO: 19 - DNA, 1116 bp, Catharanthus roseus
  source 1..1116: mol_type = genomic DNA
atggttgccacaattgattccattgaaatgcctgctcttccaacagctgtggaagcccacccaatgaaaggtggtgatgactcccacagttactcccaaaattcttgctaccagaaaggagtaattgatgcagccaaggcagtgattgttgaagcagtgaatgaaaaattggatcttgaaaataatccaatctttgatccaatcaaacctttccgcattgcagattttggatgttcaacaggaccaaatactttccacgctatgcaaaacatagtagaatcagttgaaacaaaatacaaatcacttcaaaaaacccctgaattccatgttttcttcaatgatcatgttaacaatgatttcaatgttctctttagatcccttccaccaaacagggaattttttgctgctggtgttcctggatctttctacactagagtttttcccaaaaatagcattcattttgctcattgttcttatgcacttcattggttatctaaagtgcccaaggaaattcaagataaaaattctttggcttataataagggaagaattcattacactggtactgaaaaacatgttgttaaggcttattttggtcaattccaaagagattttgaagggtttttaaaagcaagagctcaagaaattgttgttggagggttgatggtgattcaaatacctgggcttcctagtggtgaagtccttttctcaaggactggtgctggtttgcttcacttccttttgggaacttccttgatggaattggttaacaagggaatcatcaatgaagaaagtgtagattccttcaatttgcctcaatatcatccctcagttgaagacttggaaatggtgatagaaatgaacgattgtttcacaattgaaagggttggaactttacctcatcccatgaagaatttaccatttgatgttcaaaggacttctttacaagtaagagcaatcatggaatgcattctcactgaacattttggggaaaatattttggatccattatttgaaatctacaccaaaaatttgcaagagaatttccatgtttttgataaggaaattagaaaggatgcagatttgtaccttgtcttgaaacgcaagggaaattaa

(19) United States
(12) Patent Application Publication
(10) Pub. No.: US 2026/0297601 A1
(43) Pub. Date: Oct. 1, 2026

(54) METHOD OF PRODUCING IRIDOIDS

(71) Applicant: NICOVENTURES TRADING LIMITED, London (GB)

(72) Inventors: Omer BAYAZEID, Winchester (GB); Anupriya THIND, London (GB); Sara BEN KHALED, Southampton (GB)

(21) Appl. No.: 19/478,144

(22) PCT Filed: Apr. 26, 2024
(86) PCT No.: PCT/GB2024/051095

§ 371 Date: Oct. 23, 2025

(30) Foreign Application Priority Data
Apr. 28, 2023 (GB) 2306354.8

(51) Int. Cl. C12N 15/82; C07D 311/94; C07K 14/415; C12N 5/04; C12N 9/00

(52) U.S. Cl. CPC C12N 15/8243; C07D 311/94; C07K 14/415; C12N 5/04; C12N 9/00; C12N 2510/02

(57) ABSTRACT

The present disclosure relates to a composition, cells transformed with the composition, use of the composition, methods of using the composition, and products obtainable from the method.

### FIELD OF THE INVENTION

The present disclosure relates to a composition, cells transformed with the composition, use of the composition, methods of using the composition, and products obtainable from the method.

### BACKGROUND OF THE INVENTION

Iridoids are a specialised class of monoterpene found in plants and animals. When bound to glucose, as is often the case in plants, they can also be referred to as iridoid glycosides (Viljoen et al., Anti-Inflammatory Iridoids of Botanical Origin. Curr Med Chem. 2012. 19, 2104-127; Palmer et al., In vivo characterization of key iridoid biosynthesis pathway genes in catnip (Nepeta cataria). Planta. 2022. 256, 99).

Iridoids are the active compounds in many ethnobotanicals, such as species belonging to the Apocynaceae, Lamiaceae, Loganiaceae, Rubiaceae, Scrophulariaceae and Verbenaceae families, which have been traditionally used to treat inflammation. In addition to their anti-inflammatory properties, iridoids exhibit further pharmacological activities including cardiovascular, hepatoprotection, hypoglycaemic, antimutagenic, antispasmodic, anti-tumour, antiviral, immunomodulation and purgative effects (Viljoen et al., Anti-Inflammatory Iridoids of Botanical Origin. Curr Med Chem. 2012. 19, 2104-127).

### SUMMARY OF THE INVENTION

In one aspect, there is provided a composition for promoting the expression of one or more iridoids in a cell, wherein the composition comprises:
a) one or more vector components encoding one or more enzymes present in the iridoid pathway; and/or
b) one or more vector components encoding one or more transcription factors.

In one embodiment, the one or more vector components encoding one or more enzymes present in the iridoid pathway are provided in a first vector and the one or more vector components encoding one or more transcription factors are provided in a second vector.

In an alternative embodiment, the one or more vector components encoding one or more enzymes present in the iridoid pathway and the one or more vector components encoding one or more transcription factors are provided in a single vector.

In an alternative embodiment, only the one or more vector components encoding one or more enzymes present in the iridoid pathway are provided in a vector.

In an alternative embodiment, only the one or more vector components encoding one or more transcription factors are provided in a vector.

In one aspect, there is provided a plant, plant part or plant cell transformed with the vector components according to any one of the composition embodiments described herein.

In one aspect, there is provided use of the composition according to any one of the composition embodiments described herein for promoting the expression of one or more iridoids in algae, moss, or any plant, plant part, or plant cells.

In one aspect, there is provided a method of promoting the expression of one or more iridoids in a cell, comprising the step of:
a) introducing the composition according to any one of the composition embodiments described herein into a cell.

Optionally, the method further comprises the steps of:
b) extracting the one or more iridoids from the cell to produce an extract;
c) detecting the one or more iridoids in the extract; and
d) purifying the one or more iridoids from the extract.

In one aspect, there is provided an iridoid obtainable from the method according to any one of the method embodiments described herein.

### BRIEF DESCRIPTION OF THE DRAWINGS

Embodiments of the invention will now be described, by way of example only, with reference to accompanying drawings, in which:

FIG. 1 schematically shows a customized pCAmbia2300 vector encoding AmDEL and AmROS1 for plant cell lines and BY2 cells.

FIG. 2 schematically shows a customized pCAmbia2300 vector encoding AtMYB12 for plant cell lines and BY2 cells.

FIG. 3 schematically shows a customized pCAmbia2300 vector encoding AmDEL, AmROS1 and AtMYB12 for plant cell lines and BY2 cells

FIG. 4 schematically shows a customized pMBL5DL108 vector encoding AmDEL and AmROS1 for moss.

FIG. 5 schematically shows a customized pUC-GW-Amp-Cat174-nohygr vector encoding AtMYB12 for algae.

FIG. 6 schematically shows a customized pUC-GW-Amp vector encoding AmDEL and AmROS1 for algae.

FIG. 7 schematically shows a customized pCAmbia2300 vector encoding NtAN2 for BY2 cells and Nicotiana species.

FIG. 8 schematically shows a customized pCAmbia2300 vector encoding AtTT8 for BY2 cells and Nicotiana species.

FIGS. 9A-C schematically show the synthesis pathway for various iridoids. Enzymatic activity is indicated by arrows. FIG. 9A shows the early part of the synthesis pathway, from the starting point of glucose, to the terpenoid precursors Geranylgeranyl pyrophosphate, Farnesyl diphosphate and Geranyl diphosphate. FIG. 9B shows the downstream pathway of Geranyl diphosphate, which leads to FIG. 9C showing the downstream pathway of cis,trans-nepetalactol mutarotate and the resultant iridoids and secoiridoids.

FIG. 10 shows HPTLC results for Nicotiana benthamiana leaves transfected with loganin genes. Stationary Phase; Merck, HPTLC Silica gel 60 F254, Mobile phase; Chloroform-Ethanol-Methanol-0.5 M NaOH 30:15:2:1.5 (v/v). All samples were dissolved in Methanol.

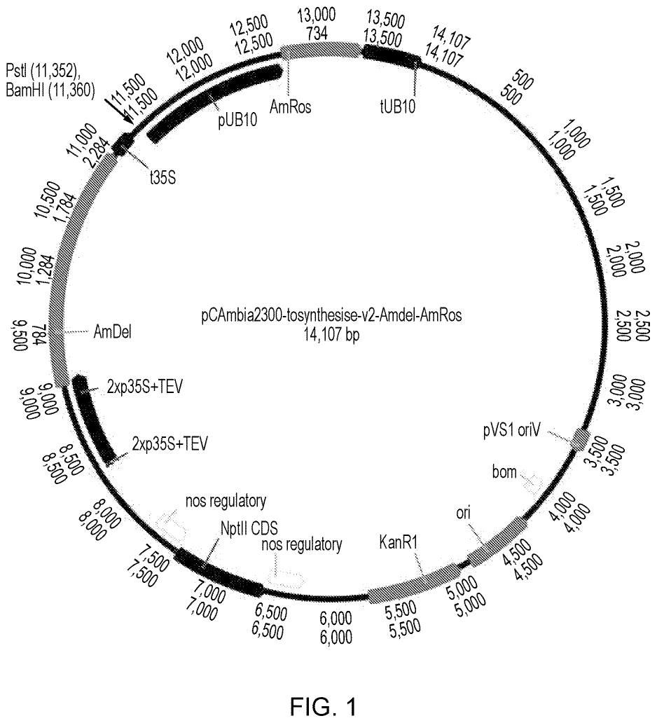

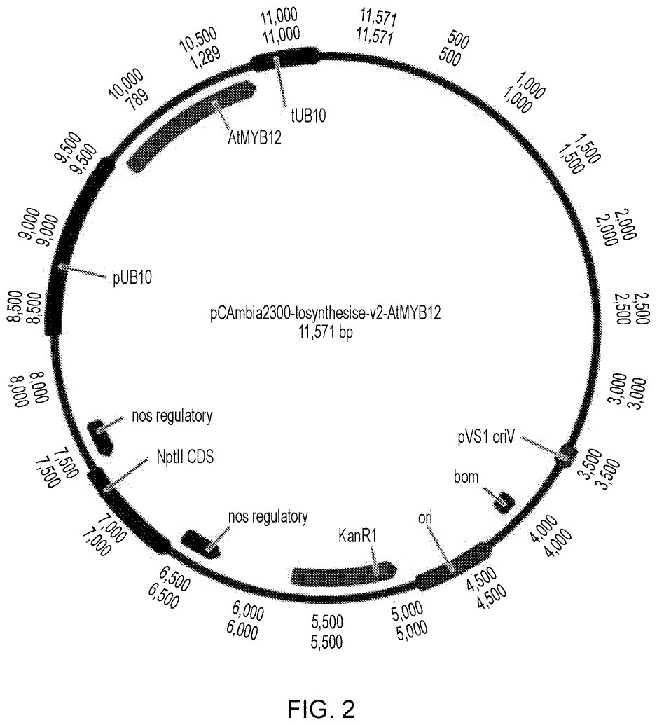

### BRIEF DESCRIPTION OF THE SEQUENCES

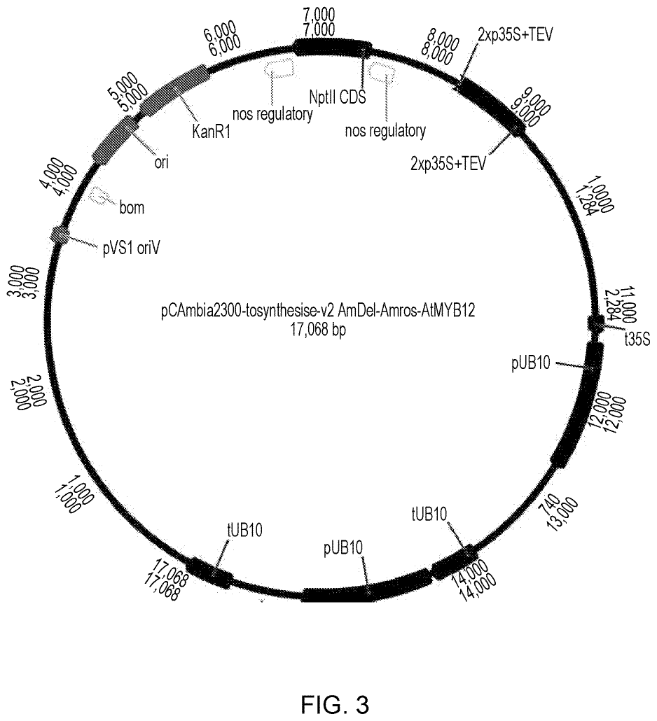

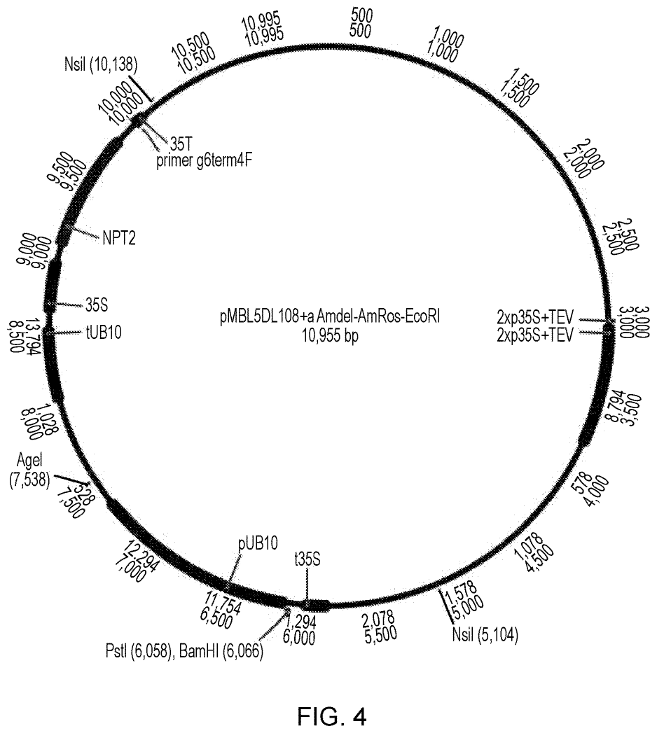

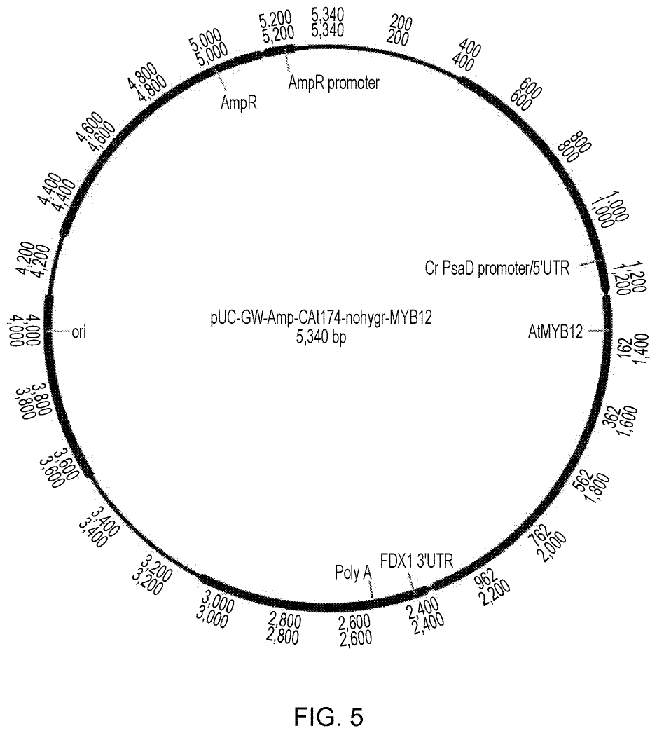

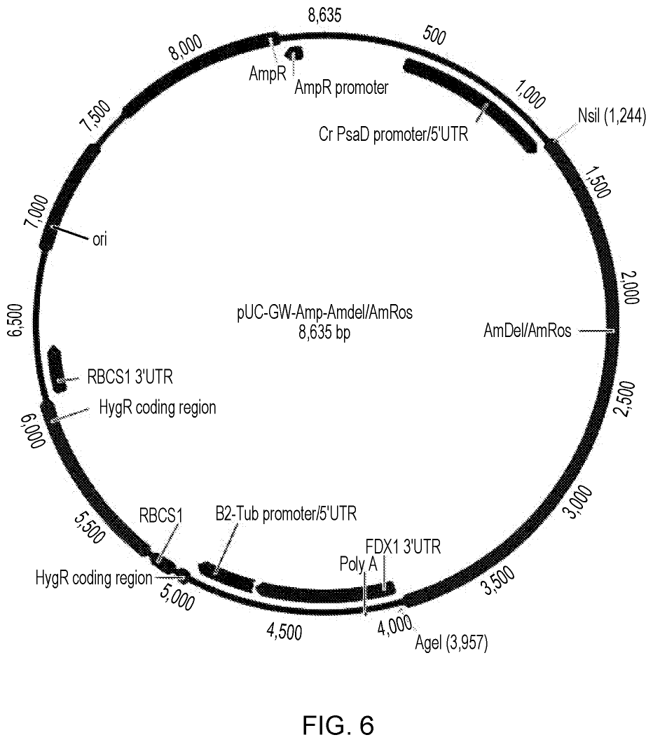

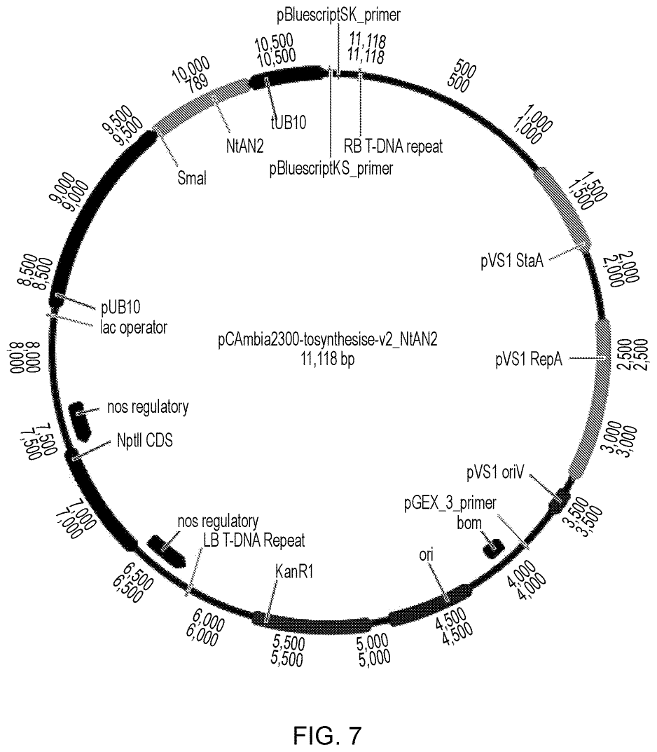

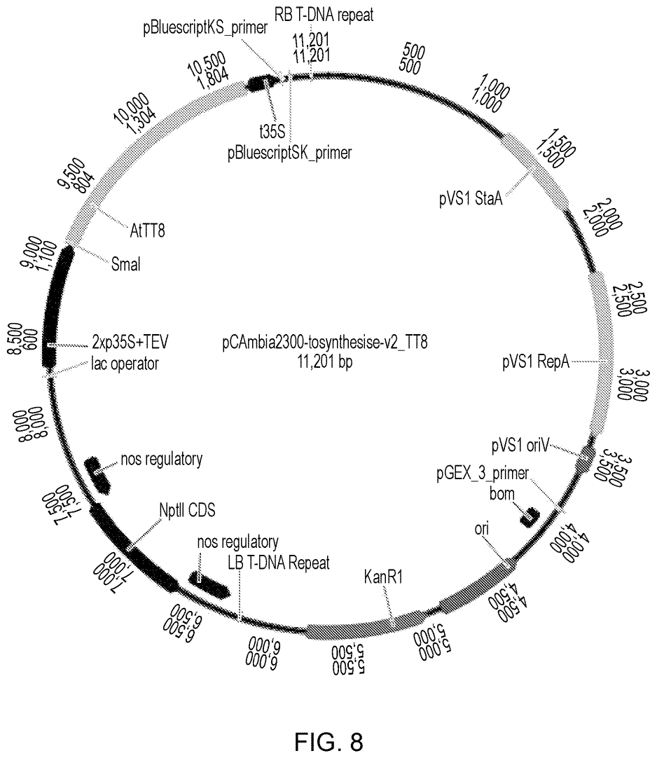

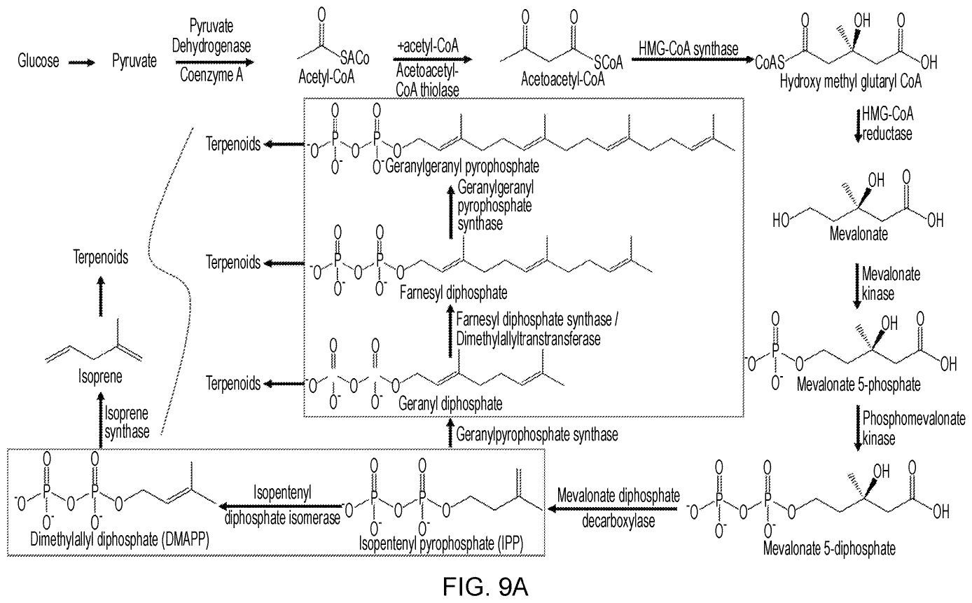

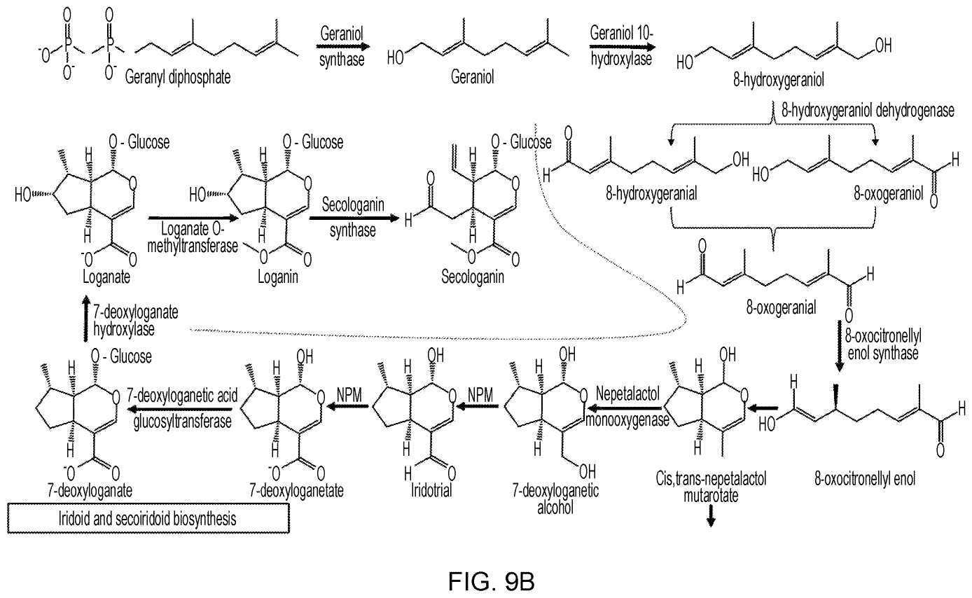

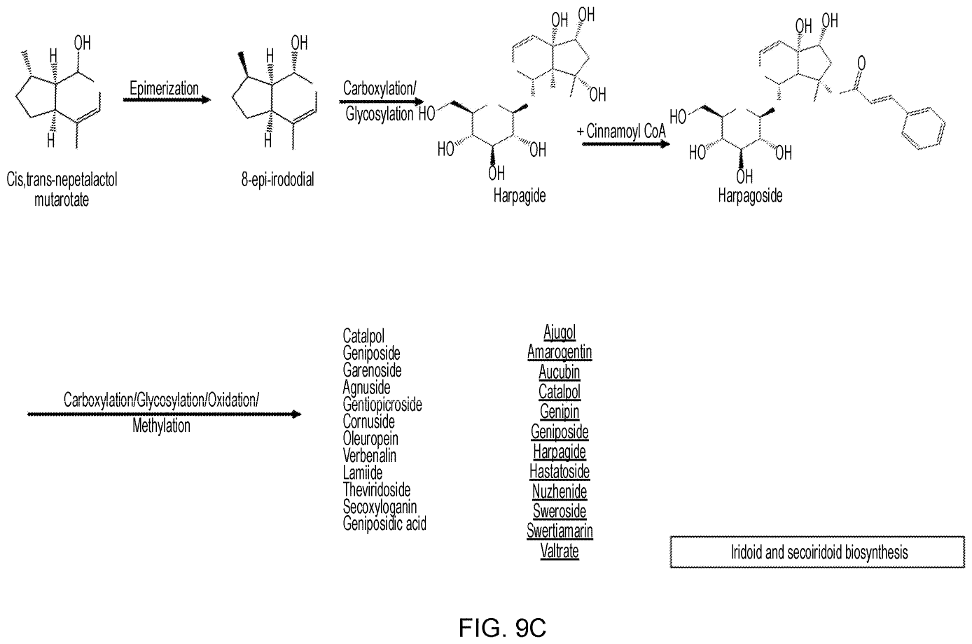

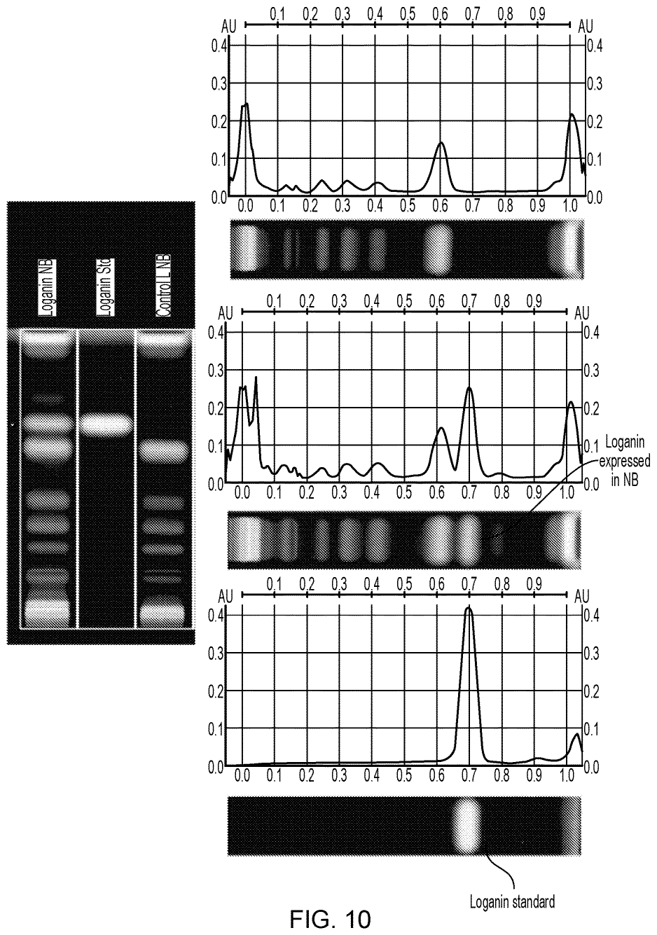

A summary of sequence identifiers used throughout the subject specification and the corresponding sequence listing is provided, wherein:
SEQ ID NO. 1 corresponds to the nucleic acid sequence of the pCAmbia2300 vector of FIG. 3, which encodes AmDEL and AmROS1.
SEQ ID NO. 2 corresponds to the nucleic acid sequence of the pCAmbia2300 vector of FIG. 4, which encodes AtMYB12.
SEQ ID NO. 3 corresponds to the nucleic acid sequence of the pCAmbia2300 vector of FIG. 5, which encodes AmDEL, AmROS1 and AtMYB12.
SEQ ID NO. 4 corresponds to the nucleic acid sequence of the pMBL5DL108 vector of FIG. 6, which encodes AmDEL and AmROS1.
SEQ ID NO. 5 corresponds to the nucleic acid sequence of the pUC-GW-Amp-Cat174-nohygr vector of FIG. 7, which encodes AtMYB12.
SEQ ID NO. 6 corresponds to the nucleic acid sequence of the pUC-GW-Amp vector of FIG. 8, which encodes AmDEL and AmROS1.
SEQ ID NO. 7 corresponds to the nucleic acid sequence of the pCAmbia2300 vector of FIG. 9, which encodes AtAN2.
SEQ ID NO. 8 corresponds to the nucleic acid sequence of the pCAmbia2300 vector of FIG. 10, which encodes AtTT8.
SEQ ID NO. 9 is a nucleic acid sequence of a vector encoding CrDXS (AB135993.1).
SEQ ID NO. 10 is a nucleic acid sequence of a vector encoding PaGPPS (ACZ57571.1).
SEQ ID NO. 11 is a nucleic acid sequence of a vector encoding CrGES (AFD64744.1).
SEQ ID NO. 12 is a nucleic acid sequence of a vector encoding CrG10H (CAC80883.1).
SEQ ID NO. 13 is a nucleic acid sequence of a vector encoding CrGOR (AHK60836.1).
SEQ ID NO. 14 is a nucleic acid sequence of a vector encoding CrISY (AFW98981.1).
SEQ ID NO. 15 is a nucleic acid sequence of a vector encoding NmMLPL (QKE59448.1).
SEQ ID NO. 16 is a nucleic acid sequence of a vector encoding CrIO (AHK60833.1).
SEQ ID NO. 17 is a nucleic acid sequence of a vector encoding Cr7-DLGT (AHK60835.1).
SEQ ID NO. 18 is a nucleic acid sequence of a vector encoding Cr7-DLH (AHK60834.1).
SEQ ID NO. 19 is a nucleic acid sequence of a vector encoding CrLAMT (ABW38009.1).

### Definitions

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. Accordingly, the terms defined immediately below are more fully defined by reference to the specification as a whole.

Where “enzymes present in the iridoid pathway” are disclosed, the skilled person will understand that the iridoid pathway may comprise any part of the pathway from the common starting point, glucose, through to the resulting iridoid end point.

Where “transcription factors present in the iridoid pathway” are disclosed, the skilled person will understand that the flavonoid pathway may encompass all of the pathway from the starting point, of the condensation of isopentenyl pyrophosphate IPP and DMAPP, through to a iridoid end product.

Where “transcription factors present in the terpenoid pathway” are disclosed, the skilled person will understand that the terpenoid pathway may encompass all of the pathway from the glucose starting point through to a terpenoid end product.

Where “transcription factors present in the flavonoid pathway” are disclosed, the skilled person will understand that the flavonoid pathway may encompass all of the pathway from the phenylalanine starting point through to a flavonoid end product. The flavonoid pathway may be divided into three subdivisions. The first stage being the phenylpropanoid synthesis pathway, the second stage being early flavonoid biosynthesis, and the third stage being late flavonoid biosynthesis.

It is to be understood that a polypeptide (synonymous with “protein” and/or “enzyme”) may be coded for by more than one nucleotide sequence due to the degeneracy of the genetic code. Therefore, a polynucleotide encoding the vector components of the invention may have at least 70%, 75%, 80%, 85%, 90%, 95%, 96%, 97%, 98% or 99% sequence identity to the disclosed nucleotide sequences (SEQ ID NO: 1, 2, 3, 4, 5, 6, 7, 8, 9, 10, 11, 12, 13, 14, 15, 16, 17, 18, or 19).

Other definitions of terms may appear throughout the specification. Before the exemplary aspects and embodiments are described in more detail, it is important to understand that this disclosure is not limited to particular aspects or embodiments described, which as such may, of course, vary. It is also to be understood that the terminology used herein is for the purpose of describing particular aspects or embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an enzyme” or “an iridoid” includes a plurality of such candidate agents and equivalents thereof known to those skilled in the art, and so forth.

### DETAILED DESCRIPTION OF THE INVENTION

Due to their various pharmacological activities, iridoids present a desirable class of compounds for use as dietary supplements and herbal medicines. However, production of iridoids from natural resources may encounter technical challenges, such as low natural yields of desirable iridoids from various plant species. Therefore, it would be advantageous to develop methods to promote the expression of iridoids of interest, such that they can be extracted in meaningful volumes for further use, in a sustainable manner.

In one aspect, there is provided a composition for promoting the expression of one or more iridoids in a cell, wherein the composition comprises:
a) one or more vector components encoding one or more enzymes present in the iridoid pathway; and optionally
b) one or more vector components encoding one or more transcription factors.

In one embodiment, the one or more vector components encoding one or more enzymes present in the iridoid pathway are provided in a first vector and the one or more vector components encoding one or more transcription factors are provided in a second vector.

In an alternative embodiment, the one or more vector components encoding one or more enzymes present in the iridoid pathway and the one or more vector components encoding one or more transcription factors are provided in a single vector.

In an alternative embodiment, only the one or more vector components encoding one or more enzymes present in the iridoid pathway are provided in a vector.

In an alternative embodiment, only the one or more vector components encoding one or more transcription factors are provided in a vector.

The skilled person will understand that the number of vectors, which comprise the vector components of the composition, will depend on the enzymes present in the iridoid pathway and the transcription factors that they wish to introduce to a cell. The claimed composition provides flexibility to tailor the encoded vector components to the suit the desired promotion of iridoids.

It will be understood that in some embodiments the one or more vector components encoding one or more enzymes present in the iridoid pathway may be present in a single vector. In other embodiments the one or more vector components encoding one or more enzymes present in the iridoid pathway may be present in more than one vector, such as two, three, four, five, six or more vectors.

It will be understood that in some embodiments the one or more vector components encoding one or more transcription factors may be present in a single vector. In other embodiments the one or more vector components encoding one or more transcription factors may be present in more than one vector, such as two, three, four, five, six or more vectors.

The number of vectors used for each vector component is independently selected such that the one or more vector encoding one or more enzymes present in the iridoid pathway components may be present in a single vector whilst the one or more vector components encoding one or more transcription factors may be present in more than one vector, and vice versa.

It will also be understood that the invention contemplates all combinations of the vector components disclosed herein. For example, the invention contemplates a first vector comprising one or more vector components encoding one or more enzymes present in the iridoid pathway, a second vector comprising one or more vector components encoding one or more transcription factors and a third vector comprising one or more vector components encoding one or more enzymes present in the iridoid pathway and one or more vector components encoding one or more transcription factors.

In one embodiment, the total nucleotide sequence length encoded by the vector components is 200 KB or less.

In one embodiment, the total nucleotide sequence length encoded by the vector components is 190 KB or less.

In one embodiment, the total nucleotide sequence length encoded by the vector components is 180 KB or less.

In one embodiment, the total nucleotide sequence length encoded by the vector components is 170 KB or less.

In one embodiment, the total nucleotide sequence length encoded by the vector components is 160 KB or less.

In one embodiment, the total nucleotide sequence length encoded by the vector components is 150 KB or less.

In one embodiment, the total nucleotide sequence length encoded by the vector components is 140 KB, 130 KB, 120 KB, 110 KB, 100 KB, 90 KB, 80 KB, 70 KB, 60 KB, 50 KB, 40 KB, 30 KB, 20 KB, 10 KB or less.

In one embodiment, the total nucleotide sequence length encoded by the vector components is 10 KB or more.

In one embodiment, the total nucleotide sequence length encoded by the vector components is 20 KB or more.

In one embodiment, the total nucleotide sequence length encoded by the vector components is 30 KB or more.

In one embodiment, the total nucleotide sequence length encoded by the vector components is 40 KB or more.

In one embodiment, the total nucleotide sequence length encoded by the vector components is 50 KB or more.

In one embodiment, the total nucleotide sequence length encoded by the vector components is 60 KB, 70 KB, 80 KB, 90 KB, 100 KB, 110 KB, 120 KB, 130 KB, 140 KB, 150 KB, 160 KB, 170 KB, 180 KB, 190 KB, 200 KB or more.

In one embodiment, the total nucleotide sequence length encoded by the vector components is between 100 KB and 150 KB.

The total nucleotide sequence length of the vector components will depend on whether they are to be inserted independently into two or more vectors, or in combination in a single vector.

The skilled person will understand that the total nucleotide sequence length of the vector components is dependent on the vector construct to which they are to be inserted and thus could be greater than 200 KB in certain circumstances.

The skilled person will also understand that the vector construct in which the vector components are contained will be selected based on the skilled persons knowledge of suitable vectors for the species of interest. The vector constructs disclosed herein are examples of suitable constructs for algae, moss and plant species. However, other vector constructs may also be appropriate and the skilled person would be aware how to select an appropriate vector construct for their intended species.

Therefore, the skilled person will use their knowledge and the information disclosed herein to determine the total sequence length and the vector construct required for efficient transformation into the species of interest.

In one embodiment, one or more vectors comprising the vector component encoding one or more enzymes present in the iridoid pathway and/or vector component encoding one or more transcription factors are added to a cell.

In one embodiment, two or more vectors comprising the vector component encoding one or more enzymes present in the iridoid pathway and/or vector component encoding one or more transcription factors are added to a cell.

In one embodiment, three or more vectors comprising the vector component encoding one or more enzymes present in the iridoid pathway and/or vector component encoding one or more transcription factors are added to a cell.

In one embodiment, four or more vectors comprising the vector component encoding one or more enzymes present in the iridoid pathway and/or vector component encoding one or more transcription factors are added to a cell.

In one embodiment, five or more vectors comprising the vector component encoding one or more enzymes present in the iridoid pathway and/or vector component encoding one or more transcription factors are added to a cell.

In one embodiment, six or more vectors comprising the vector component encoding one or more enzymes present in the iridoid pathway and/or vector component encoding one or more transcription factors are added to a cell.

In one embodiment, seven or more vectors comprising the vector component encoding one or more enzymes present in the iridoid pathway and/or vector component encoding one or more transcription factors are added to a cell.

In one embodiment, eight or more vectors comprising the vector component encoding one or more enzymes present in the iridoid pathway and/or vector component encoding one or more transcription factors are added to a cell.

In one embodiment, nine or more vectors comprising the vector component encoding one or more enzymes present in the iridoid pathway and/or vector component encoding one or more transcription factors are added to a cell.

In one embodiment, ten or more vectors comprising the vector component encoding one or more enzymes present in the iridoid pathway and/or vector component encoding one or more transcription factors are added to a cell.

In one embodiment, up to ten vectors comprising the vector component encoding one or more enzymes present in the iridoid pathway and/or vector component encoding one or more transcription factors are added to a cell.

In one embodiment, the cell is a plant cell.

In a further embodiment, the plant cell is an algae cell, moss cell, or any cell or cell line derived from the genus Nicotiana.

In yet a further embodiment, the cell or cell line derived from the genus Nicotiana is selected from the group comprising or consisting of Nicotiana tabacum, Nicotiana benthamiana, or BY-2 cells.

In an alternative embodiment, the cell is a yeast cell or a bacterial cell.

The skilled person will understand that the type of vector selected will depend on its intended purpose. The vector may be any suitable type of vector such as a plasmid, cosmid, phage, or viral vector.

In one embodiment, the vector comprising the vector component encoding one or more enzymes present in the terpenoid pathway and/or vector component encoding one or more transcription factors is a plasmid vector.

In a further embodiment, the plasmid vector is a plasmid binary vector.

We have found that by tailoring the selection of enzymes present in the iridoid pathway to be encoded by the vector component encoding one or more enzymes present in the iridoid pathway, and/or transcription factors encoded by the vector component encoding one or more transcription factors present in the iridoid pathway, expression of specific iridoids can be promoted in a transfected cell.

For ease of reference, these and further aspects of the present invention are now discussed under appropriate section headings. However, the teachings under each section are not necessarily limited to each particular section.

Enzymes present in the iridoid pathway As described herein, the composition for promoting the expression of one or more iridoids comprises one or more vector components encoding one or more enzymes present in the iridoid pathway.

In a further embodiment, the one or more enzymes present in the iridoid pathway comprise one or more of the group comprising or consisting of 1-deoxy-D-xylulose 5-phosphate synthase (DXS), geranyl pyrophosphate synthase (GPPS), geraniol synthase (GES), geraniol 10-hydroxylase (G10H), 8-hydroxygeraniol oxidoreductase (GOR), iridoid synthase (ISY), major-latex protein-like enzyme (MLPL), iridoid oxidase (10), 7-deoxyloganetic acid glucosyl transferase (7-DLGT), 7-deoxyloganic acid hydroxylase (7-DLH), and loganic acid O-methyltransferase (LAMT), Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, Cinnamoyl CoA.

In one embodiment, the one or more vector components, encoding one or more enzymes present in the iridoid pathway, encodes two or more enzymes present in the iridoid pathway.

In a further embodiment, the two or more enzymes present in the iridoid pathway comprise two or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components, encoding one or more enzymes present in the iridoid pathway, encodes three or more enzymes present in the iridoid pathway.

In a further embodiment, the three or more enzymes present in the iridoid pathway comprise three or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes four or more enzymes present in the iridoid pathway.

In a further embodiment, the four or more enzymes present in the iridoid pathway comprise four or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components, encoding one or more enzymes present in the iridoid pathway, encodes five or more enzymes present in the iridoid pathway.

In a further embodiment, the five or more enzymes present in the iridoid pathway comprise five or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes six or more enzymes present in the iridoid pathway.

In a further embodiment, the six or more enzymes present in the iridoid pathway comprise six or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes seven or more enzymes present in the iridoid pathway.

In a further embodiment, the seven or more enzymes present in the iridoid pathway comprise seven or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes eight or more enzymes present in the iridoid pathway.

In a further embodiment, the eight or more enzymes present in the iridoid pathway comprise eight or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes nine or more enzymes present in the iridoid pathway.

In a further embodiment, the nine or more enzymes present in the iridoid pathway comprise nine or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes ten or more enzymes present in the iridoid pathway.

In a further embodiment, the ten or more enzymes present in the iridoid pathway comprise ten or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes eleven or more enzymes present in the iridoid pathway.

In a further embodiment, the eleven or more enzymes present in the iridoid pathway comprise eleven or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

In one embodiment, the one or more vector components encoding one or more enzymes present in the iridoid pathway, encodes 12, 13, 14, 15, 16, 17, 18, 19, 20, or more enzymes present in the iridoid pathway.

In a further embodiment, the 12, 13, 14, 15, 16, 17, 18, 19, 20, or more enzymes present in the iridoid pathway comprise 12, 13, 14, 15, 16, 17, 18, 18, 20, or more of the group comprising or consisting of DXS, GPPS, GES, G10H, GOR, ISY, MLPL, 10, 7-DLGT, 7-DLH, LAMT, Pyruvate Dehydrogenase, Coenzyme A, acetyl-CoA, Acetoacetyl-CoA thiolase, HMG-CoA synthase, HMG-CoA reductase, Mevalonate kinase, Phosphomevalonate kinase, Mevalonate diphosphate decarboxylase, Isopentenyl diphosphate isomerase, Isoprene synthase, Geranylpyrophosphate synthase, Farnesyl diphosphate synthase, Dimethylallyltranstransferase, Geranylgeranyl pyrophosphate synthase, Geraniol synthase, Geraniol 10-hydroxylase, 8-hydroxygeraniol dehydrogenase, 8-oxocitronellyl enol synthase, nepetalactol monooxygenase, NPM, 7-deoxyloganetic acid glucosyltransferase, 7-deoxyloganate hydroxylase, loganate O-methyltransferase, secologanin synthase, and Cinnamoyl CoA.

The skilled person will be aware that the maximum number of enzymes encoded by the vector component encoding one or more enzymes present in the iridoid pathway will be dependent on the constraints of the vector construct chosen. E.g. the total sequence length that the vector construct can support.

Described below are non-limiting examples of combinations of enzymes encoded by the vector components, which may be chosen by the skilled person to promote the expression of desired iridoids in cells.

In one embodiment, the one or more enzymes present in the iridoid pathway are DXS, GPPS, GES, G10H, GOR, ISY, MLPL, 10, 7-DLGT, 7-DLH, and LAMT.

The skilled person understands that enzymes present in the terpenoid pathway may be isolated from a variety of different plant species. Thus, a plurality of different isoforms may exist for each enzyme described herein.

In selecting the combination of enzymes present in the iridoid pathway, the skilled person will use their knowledge of the iridoid pathway to select appropriate enzymes to reach the desired iridoid. Examples of the various enzymes required to reach different iridoid end products can be seen in FIGS. 9A-C.

### Transcription Factors

As described herein, the composition for promoting the expression of one or more iridoids optionally comprises one or more vector components encoding one or more transcription factors.

In one embodiment, the one or more transcription factors are selected from the group comprising or consisting of myeloblastosis (MYB) type transcript ion factors and/or basic helix-loop-helix (bHLH) type transcription factors.

In a further embodiment, the one or more transcription factors are independently derived from a plant species.

The skilled person will understand that the transcription factors listed above and below may be derived from different plant species. Therefore, the listed transcription factors are intended to encompass those derived from any species including, but not necessarily limited to the prefixes At, Vv, Ib, Gh, Gm, Md, Gt, Ms, Co, Ej, Mg, Pc, Ppy, Pb, Par, Pi, Pdm, Pav, Pcr, Pcf, Pd, Ppr, Ps, Fa, Fv, Rh, Ri, Hv, Bo, Mr, Eg, Es, In, Dk, Mt, Lh, Lj, Zm, Pp, Cs, Og, Py, Pt, Ptr, Pto, St, Ta, Nt, and Le.

In one yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the one or more vector components encoding one or more transcription factors, encode two or more transcription factors.

In one embodiment, the two or more transcription factors are selected from the group comprising or consisting of myeloblastosis (MYB) type transcription factors and/or basic helix-loop-helix (bHLH) type transcription factors.

In a further embodiment, the two or more transcription factors are independently derived from a plant species.

In one yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the one or more vector components encoding one or more transcription factors, encode three or more transcription factors.

In one embodiment, the three or more transcription factors are selected from the group comprising or consisting of myeloblastosis (MYB) type transcription factors and/or basic helix-loop-helix (bHLH) type transcription factors.

In a further embodiment, the three or more transcription factors are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

The skilled person will be aware that the maximum number of transcription factors encoded by the vector component encoding one or more transcription factors will be dependent on the constraints of the vector construct chosen. E.g. the total sequence length that the vector construct can support.

In embodiments where each transcription factor is independently derived from a plant species, it should be noted that each transcription factor need not necessarily be derived from the same plant species.

MYB proteins are transcription factors found in all eukaryotes. However, whilst only a few unique MYBs are encoded in unikonts (the phylogenetic group comprising animals and fungi), there are up to ~200 unique MYB genes found in the plant lineage. Of the MYB proteins present in plants, R2R3 MYB transcription factors are the most widely encoded, with many plant species encoding>100 unique R2R3 MYB.

In one embodiment, the MYB type transcription factor is a member of the R2R3 MYB transcription factor family.

In one embodiment, the MYB type transcription factor is one or more of MYB12, AN2, and ROS1.

In one embodiment, the MYB type transcription factor is MYB12.

In one embodiment, the MYB type transcription factor is AN2.

In one embodiment, the MYB type transcription factor is ROS1

bHLH type transcription factors are so named due to their highly conserved alkaline/helix-loop-helix domain. Originally identified in animals, they have also been found in plants and are involved in diverse functions including plant growth and development, via the functions of DNA binding and dimerization.

In one embodiment, the bHLH type transcription factor is one or more of Triterpene Saponin Biosynthesis Activating Regulator 1 (TSAR1), Triterpene Saponin Biosynthesis Activating Regulator 2 (TSAR2), Triterpene Saponin Biosynthesis Activating Regulator 3 (TSAR3), DEL, TT8, iridoid synthesis 1, iridoid synthesis 2, or iridoid synthesis 3.

In one embodiment, the bHLH type transcription factor is TSAR1 In one embodiment, the bHLH type transcription factor is TSAR2.

In one embodiment the bHLH type transcription factor is TSAR3.

In one embodiment, the bHLH type transcription factor is TSAR1, TSAR2 and/or TSAR3.

In one embodiment, the bHLH type transcription factor is DEL.

In one embodiment the bHLH type transcription factor is TT8.

In one embodiment, the bHLH type transcription factor is iridoid synthesis 1.

In one embodiment, the bHLH type transcription factor is iridoid synthesis 2.

In one embodiment, the bHLH type transcription factor is iridoid synthesis 3.

In one embodiment, the one or more transcription factors are one or more transcription factors present in the terpenoid pathway.

In one embodiment, the one or more transcription factors are one or more transcription factors present in the flavonoid pathway.

In one embodiment, the one or more transcription factors are one or more transcription factors present in the iridoid pathway.

In one embodiment, the one or more transcription factors are one or more transcription factors present in the iridoid pathway, and/or terpenoid pathway, and/or flavonoid pathway.

In one embodiment, the one or more transcription factors present in the iridoid pathway, and/or terpenoid pathway, and/or flavonoid pathway are selected from the group comprising or consisting of MYB12, AN2, ROS1, DEL, TT8, TSAR1, TSAR2, TSAR3, iridoid synthesis 1, iridoid synthesis 2, and iridoid synthesis 3.

In one embodiment, the one or more transcription factors present in the iridoid pathway, are selected from the group comprising or consisting of iridoid synthesis 1, iridoid synthesis 2, and iridoid synthesis 3.

In one embodiment, the one or more transcription factors present in the terpenoid pathway are selected from the group comprising or consisting of TSAR1, TSAR2, and TSAR3.

In one embodiment, the one or more transcription factors present in the flavonoid pathway are selected from the group comprising or consisting of MYB12, AN2, ROS1, DEL, and TT8.

The skilled person will understand that different plant species have their own transcription factors present in the iridoid pathway. Therefore, the lists of transcription factors present in the iridoid pathway provided above are non-limiting and the skilled person would select the most appropriate transcription factors based on the plant species of interest.

Iridoids obtainable from the composition As has already been disclosed, iridoids are a group of natural products found in plants and animals, with useful pharmaceutical activity. Therefore, iridoids present a group of commercially attractive compounds.

As described herein, the composition promotes the expression of one or more iridoids.

In one embodiment, the one or more iridoids are one or more plant-derived iridoids.

In one embodiment, the one or more iridoids are one or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside III, opuloside IV, opuloside X, opulus iridoid II, opulus iridoid III, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-β-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-0-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the one or more iridoids may be any one or more of the iridoids listed in FIGS. 9A-C.

In one embodiment, the one or more iridoids are one or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the one or more iridoids is loganin, or a derivative thereof.

In one embodiment, the one or more iridoids is harpagoside, or a derivative thereof.

In one embodiment, the composition promotes the expression of two or more iridoids.

In one embodiment, the two or more iridoids are two or more plant-derived iridoids.

In one embodiment, the two or more iridoids are two or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside III, opuloside IV, opuloside X, opulus iridoid II, opulus iridoid III, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-p-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-0-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the two or more iridoids may be any two or more of the iridoids listed in FIGS. 9A-C.

In one embodiment, the two or more iridoids are two or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein Ill, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside Ill, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the two or more iridoids are loganin, or a derivative thereof and at least one other iridoid.

In one embodiment, the two or more iridoids are harpagoside, or a derivative thereof and at least one other iridoid.

The skilled person will understand that due to the nature of catalysis reactions, not all intermediate metabolites will be catalysed to form the next metabolite. The selected combinations of enzymes and transcription factors present in the iridoid pathway will promote the expression of particular iridoids. However, intermediate metabolites of the pathway may also be present.

Therefore, a plurality of iridoid metabolites may be expressed in the cell transfected with the vector components described herein.

In certain embodiments, the iridoid obtained and the combination of enzymes and transcription factors present in the iridoid pathway to obtain the iridoid are as recited in Table 1.

TABLE 1
provides non-limiting examples of combinations of
enzymes and transcription factors present in the
iridoid pathway, encoded by the vector components
as described herein, used to obtain iridoids.

| | | Transcription factors |
|---|---|---|
| | Enzymes present in the | present in the iridoid |
| Iridoid obtained | iridoid pathway [optional] | pathway [optional] |
| Loganin | NA | [MYB12/AN2/TT8] |
| Harpagoside | NA | [AN2] |

### Plant Products

As described herein, in one aspect there is provided a plant, plant part or plant cell transformed with the vector components according to any one of the vector composition embodiments described herein.

In one embodiment, the plant, plant part or plant cell is algae, moss or any plant, plant part or plant cell derived from the genus Nicotiana.

In a further embodiment, the plant, plant part or plant cell derived from the genus Nicotiana is selected from the group comprising or consisting of Nicotiana tabacum, Nicotiana benthamiana, or BY-2 cells.

### Use

As described herein, in one aspect there is provided use of the composition according to any one of the composition embodiments described herein for promoting the expression of one or more iridoids in algae, moss, or any plant, plant part, or plant cells.

In one embodiment, the plant cells are a cell line derived from plants.

In one embodiment, the plant, plant part, or plant cells are derived from the genus Nicotiana.

In yet a further embodiment, the plant, plant part or plant cell derived from the genus Nicotiana is selected from the group comprising or consisting of Nicotiana tabacum, Nicotiana benthamiana, or BY-2 cells.

In an alternative embodiment, the expression of one or more iridoids is promoted in yeast cells and/or bacterial cells.

In one embodiment, the composition promotes the expression of one or more iridoids.

In one embodiment, the one or more iridoids are one or more plant-derived iridoids.

In one embodiment, the one or more iridoids are one or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein Ill, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside III, opuloside IV, opuloside X, opulus iridoid II, opulus iridoid III, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-p-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-0-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the one or more iridoids may be any one or more of the iridoids listed in FIGS. 9A-C.

In one embodiment, the one or more iridoids are one or more of the group comprising or consisting of loganin, secologanin, catalpol, geniposide, garenoside, agnuside, gentiopicroside, cornuside, oleuropein, demethyloleuropein, oleacein, verbenalin, Iamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, Iigstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the one or more iridoids is loganin, or a derivative thereof.

In one embodiment, the one or more iridoids is harpagoside, or a derivative thereof.

In one embodiment, the composition promotes the expression of two or more iridoids.

In one embodiment, the two or more iridoids are two or more plant-derived iridoids.

In one embodiment, the two or more iridoids are two or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-p-trans-coumaroyl-1S-dihydromonotropein, 10-p-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein Ill, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside III, opuloside IV, opuloside X, opulus iridoid II, opulus iridoid III, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β3-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-p-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-0-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the two or more iridoids may be any two or more of the iridoids listed in FIGS. 9A-C.

In one embodiment, the two or more iridoids are two or more of the group comprising or consisting of loganin, secologanin, catalpol, geniposide, garenoside, agnuside, gentiopicroside, cornuside, oleuropein, demethyloleuropein, oleacein, verbenalin, Iamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, Iigstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the two or more iridoids are loganin, or a derivative thereof and at least one other iridoid.

In one embodiment, the two or more iridoids are harpagoside, or a derivative thereof and at least one other iridoid.

### Method

As described herein, in one aspect there is provided a method of promoting the expression of one or more iridoids in a cell, comprising the step of:
a) introducing the composition according to any one of the composition embodiments described herein into a cell.

In one embodiment, the method further comprises:
b) extracting the one or more iridoids from the cell to produce an extract.

In an additional embodiment, the method further comprises:
c) detecting the one or more iridoids in the extract.

In an additional embodiment, the method further comprises:
d) purifying the one or more iridoids from the extract.

In one embodiment, the method further comprises one or more of:
b) extracting the one or more iridoids from the cell to produce an extract;
c) detecting the one or more iridoids in the extract; and
d) purifying the one or more iridoids from the extract.

In one embodiment, the method further comprises:
b) extracting the one or more iridoids from the cell to produce an extract;
c) detecting the one or more iridoids in the extract; and
d) purifying the one or more iridoids from the extract.

In a further embodiment, the one or more iridoids are purified using Flash and preparative MPLC, HPTLC or LC/MS.

In one embodiment, the cell is a plant cell, yeast cell and/or bacteria cell.

In a further embodiment, the plant cell is an algae cell, moss cell, or any cell or cell line derived from the genus Nicotiana.

In yet a further embodiment, the cell or cell line derived from the genus Nicotiana is selected from the group comprising or consisting of Nicotiana tabacum, Nicotiana benthamiana, or BY-2 cells.

In one embodiment, the composition promotes the expression of one or more iridoids.

In one embodiment, the one or more iridoids are one or more plant-derived iridoids.

In one embodiment, the one or more iridoids are one or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β3-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-p-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-0-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the one or more iridoids may be any one or more of the iridoids listed in FIGS. 9A-C.

In one embodiment, the one or more iridoids are one or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β3,7-β-epoxygaertneroside, 6-β3,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein Ill, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the one or more iridoids is loganin, or a derivative thereof.

In one embodiment, the one or more iridoids is harpagoside, or a derivative thereof.

In one embodiment, the composition promotes the expression of two or more iridoids.

In one embodiment, the two or more iridoids are two or more plant-derived iridoids.

In one embodiment, the two or more iridoids are two or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β,7-β-epoxygaertneroside, 6-β,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β3-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-p-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-O-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the two or more iridoids may be any two or more of the iridoids listed in FIGS. 9A-C.

In one embodiment, the two or more iridoids are two or more of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β3,7-β-epoxygaertneroside, 6-β3,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein Ill, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside Ill, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the two or more iridoids are loganin, or a derivative thereof and at least one other iridoid.

In one embodiment, the two or more iridoids are harpagoside, or a derivative thereof and at least one other iridoid.

### Products

As described herein, in one aspect there is provided an iridoid obtainable from the method according to any one of the method embodiments described herein.

In one embodiment, the iridoid is a plant-derived iridoid.

In one embodiment, the iridoid is any one of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β3,7-β-epoxygaertneroside, 6-β3,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein III, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside III, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, 8-O-Acetylharpagide, 6′-O-Acetylgeniposide, 6′-O-Acetyl-3″-methoxygaertneroside, 10-O-Acetylpatrinoside-aglycone-11-O-[4″-O-acetyl-α-L-rhamnopyranosyl-(1→2)-β-D-glucopyranoside], 7-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[4″-O-acetyl-α-L-rhamnopyranosyl-(1→2)-β-D-ribohexo-3-ulopyranoside], 10-O-Acetylpatrinoside-aglycone-11-O-[α-L-rhamnopyranosyl-(1→2)-β-D-ribohexo-3-ulopyranoside], Acevaltrate, Adenosmoside, Alatenoside (C-7α-OH epimer), Alatinoside (C-7β3-OH epimer), Allamanoid, Amphicoside, Buddlejosides B, Buddlejosides As, Buddlejosides A₂, 2′-O-Caffeoylloganic acid, 7-epi-7-O-(E)-Caffeoylloganic acid, Catarractoside (=6-O-Rhamnopyranosylasystasioside E), 6″-O-trans-Cinnamoylgenipin gentiobioside, 6′-O-Cinnamoylharpagide, 6-O-[3″-(E)-Cinnamoyl-α-L-rhamnopyranosyl]-glutinoside, Cocculoside, 6-O-cis-p-Coumaroylcatalpol, 6″-O-trans-p-Coumaroylgenipin gentiobioside, 6′-O-trans-p-Coumaroylgeniposide, 6′-O-trans-p-Coumaroylgeniposidic acid, 2′-O-E-p-Coumaroylloganic acid, 2′-O-Z-p-Coumaroylloganic acid, 10-O-(E)-p-Coumaroylmelittoside, 15-Demethylisoplumieride, Derwentioside A (=6-O-Benzoylaucubin), Derwentioside B (=6-O-p-Hydroxybenzoylaucubin), Derwentioside C(=6-O-Vanilloylaucubin), Didrovaltrate acetoxyhydrin, Diffusoside A, Diffusoside B, (1R)-1,5-Dihydroxy-3,8-epoxyvalechlorine, 6′-(3,4-Dimethoxycinnamoyl)-ajugol, 6′-(3,4-Dimethoxycinnamoyl)-harpagide, Dunalianoside I, 8-Epiloganic acid, (1R)-3,8-Epoxy-1-O-ethyl-5-hydroxyvalechlorine, (1 S)-3,8-Epoxy-1-O-ethyl-5-hydroxyvalechlorine, Eurostoside, Excelside A, Excelside B, Fagraldehyde, Floribundane A, Floribundane B, GEIR-1, GEIR-2, GEIR-3, GI-3, GI-5, Glucologanin, 2′-O-β-D-Glucopyranosyl-6′-O-(p-methoxycinnamoyl)-harpagide, 6β-O-β-D-Glucosylpaederosidic acid, Griffithoside A, Griffithoside B, Griffithoside C, GRIR-1, 5-Hydroxydidrovaltrate, 2′-O-p-Hydroxylbenzoylloganic acid, (7″R)-10-Hydroxy-7″-methoxyoleuropein, (7″S)-10-Hydroxy-7″-methoxyoleuropein, Incarvillic acid, Isoplumericin, (IR)-1-Isopropyloxygenipin, Jatamanin A, Jatamanin B, Jatamanin C, Jatamanin D, Jatamanin E, Jatamanin F, Jatamanin G, Jatamanin H, Jatamanin I, Jatamanin J, Jatamanin K, Jatamanin L, Jatamanin M, Jatamanvaltrate A, Jatamanvaltrate B, Jatamanvaltrate C, Jatamanvaltrate D, Jatamanvaltrate E, Jatamanvaltrate F, Jatamanvaltrate G, Jatamanvaltrate H, Jatamanvaltrate I, Jatamanvaltrate J, Jatamanvaltrate K, Jatamanvaltrate L, Jatamanvaltrate M, Javanicoside A, Javanicoside B, Kankanoside A, Kankanoside L, Kankanoside M, Kankanoside N, Kutkoside, Lamalbid, Lamiridosin A, Lamiridosin B, Ligustrohemiacetal A, Ligustrohemiacetal B, Longifolioside A, Longifolioside B, Lonijaposide A, Lonijaposide B, Lonijaposide C, 6′-O-(p-Methoxycinnamoyl)-harpagide, E-6-O-p-Methoxycinnamoyl scandoside methyl ester, 9-epi-6α-Methoxygeniposidic acid, Minecoside, Mussaenosidic acid, Muzhenide, Naucledal, cis-Nepetalactone, trans-Nepetalactone, Oleoside dimethyl ester, Oreosolenoside, Patrinoside-aglycone-11-O-[4″-O-acetyl-α-L-rhamnopyranosyl-(1→2)-β-D-ribohexo-3-ulopyranoside], Patrinoside-aglycone-11-O-2′-deoxy-β-glucopyranoside, Patriscadoid I, Patriscadoid II, Phloyoside 1, Phlomiol, Picogentioside I, Picroside 1, Picroside II, Pirosecoside I, Pirosecoside II, Plumericin, Pulchelloside 1, Safghanoside D, Shanzhiside, Shanzhiside methyl ester, Sanshiside D, Scyphiphin A₁, Scyphiphin A₂, Scyphiphin B₁, Scyphiphin B₂, 6″-0-trans-Sinapoylgenipin gentiobioside, 10-(6-O-trans-Sinapoylglucopyranosyl)gardendiol, 11-(6-O-trans-Sinapoylglucopyranosyl)-gardendiol, Specioside, 10-O-Succinoylgeniposide, Swerilactone A, Swerilactone B, Swerilactoside A, Swerilactoside B, Swerilactoside C, Syrveoside A (=6′-O-(6,7-Dihydrofoliamenthoyl)-8-epi-kingisidic acid), Syrveoside B (=6′-O-(Menthiafoloyl)-8-epi-kingisidic acid), 2′,3′,6′-Tri-O-acetyl-4′-O-trans-p-(O-β-D-glucopyranosyl)-coumaroyl-7-ketologanin, Triohima A, Triohima B, Triohima C, Valeriotetrate C, Valtrate, IVHD-valtrate, 10-O-Vanilloylgeniposidic acid, Verminoside, Versibirioside (6-O-[3″-(E)-Cinnamoyl-α-L-rhamnopyranosyl]-glutinoside), Volvaltrate A, Volvaltrate B, or a derivative thereof.

The skilled person will understand that the iridoid may be any one of the iridoids listed in FIGS. 9A-C.

In one embodiment, the iridoid is any one of the group comprising or consisting of loganin, secologanin, catalpol, geniposide, garenoside, agnuside, gentiopicroside, cornuside, oleuropein, demethyloleuropein, oleacein, verbenalin, lamiide, theviridoside, secoxyloganin, geniposidic acid, ajugol, amarogentin, aucubin, genipin, harpagide, harpagoside, hastatoside, nuzhenide, sweroside, swertiamarin, valtrate, β-(3,4-dihydroxyphenyl)ethanol, deoxyloganic acid lauryl ester, ligstroside, oleocanthal, asperulosidic acid, deacetyloasperulosidic acid, asperuloside, deacetyloasperuloside, dehydromethoxygaertneroside, epi-dihydrocornin, 6α-hydroxyadoxoside, citrifolinin B (epimers A and B), 6b,7β-epoxy-8-epi-splendoside, morindacin, loganic acid, monotropein, scandoside methyl ester, 6-O-acetylscandoside, 9-epi-6-methoxygeniposidic acid, rhodolatouside, 6-hydroxyadoxoside, yopaaoside C, 6-epi-dihydrocornin, 10-O-acetylomonotropein, citrifoside, morofficinaloside, morindolide, umbellatolide A, umbellatolide B, tinctoroide, citrifolinoside A, gaertneroside, 6′-O-acetylgaertneroside, 3″-methoxygaertneroside, 6′-O-acetyl-3″-methoxygaertneroside, dehydrogaertneroside, morinipticoside, gaertneric acid, 6-β3,7-β-epoxygaertneroside, 6-β3,7-β-epoxy-3″-methoxy-gaertneroside, citrifolinoside B, citrifolinin A-1, oruwacin, borreriagenin, 4-epi-borreriagenin, 10-methoxyfermiloside, longifolide A, longifolide B, morintoside A, morintoside B, moludicin, daphylloside, methoxymorindic acid, methyl methoxymorindonate, M-L-2-3, ML-F52, 10-β-trans-coumaroyl-1S-dihydromonotropein, 10-β-cis-coumaroyl-1S-dihydromonotropein, depside, 2-O-(3,4-dihydroxybenzoyl)-2,4,6-trihydroxyphenylmethylacetate, p-coumaroyl monotropein I, p-coumaroyl monotropein II, p-coumaroyl monotropein Ill, p-coumaroyl monotropein IV, 6,7-dihydromonotropein (splendoside), deacetylasperulosidic acid, scandoside, 7,8-dihyroiridoid, p-coumaroyl-monotropein derivative 1, p-coumaroyl-monotropein derivative 2, p-coumaroyl-6,7-dihydromonotropein, p-coumaroyl dihydromonotropein I, p-coumaroyl dihydromonotropein II, p-coumaroyl monotropein hexoside, p-coumaroyl-deacetylasperulosidic acid derivative 1, p-coumaroyl-deacetylasperulosidic acid derivative 2, p-coumaroyl-scandoside derivative 1, p-coumaroyl-scandoside derivative 2, morroniside, 7-O-methyloganic acid, cornuside A, cornuside B, cornuside C, cornuside D, cornuside E, cornuside F, cornuside G, cornuside H, cornuside I, cornuside J, cornuside K, cornuside L, cornuside M, cornuside N, cornuside 0, cornuside I, cornuside II, cornuside Ill, cornuside IV, 8-epi-loganin, cornel iridoid glycoside, cornusfural A, cornusfural B, cornusfural C, 7-α-O methylmorroniside, 7-β-O-methylmorroniside, 7-α-O-ethylmorroniside, 7-β-O-ethylmorroniside, 7-β-O-dimethyl butanedioate morroniside, (7R)-n butyl morroniside, 7-α-morroniside, 7-β-morroniside, 7-O-cinnamoylmorroniside, logmalicid A, logmalicid B, cornusfuroside A, logmalicid B, logmalicid C, logmalicid D, eriobioside, 7-dehymorroniside, williamoside D, b-dihydrocornin, 10-hydroxyhastatoside, cornifin B, cornusphenoside A, cornusphenoside B, cornusphenoside C, cornusphenoside D, loganic acid isomer 1, loganic acid isomer 2, cornuside 2, catalposide, 7-epi-loganic acid, 8-epi-loganic acid, pentosyl-loganic acid, loganic acid 7-O-pentoside, 7-epi-loganic acid 7-O-pentoside, pentosyl-loganin, pentosyl-sweroside, loganin 7-O-pentoside, 7-epi-loganin, 7-epi-loganin 7-O-pentoside sweroside, secologanate, viburtinoside derivative IV, viburtinoside derivative V, opuloside I, opuloside II, opuloside Ill, opuloside IV, opuloside X, opulus iridoid II, opulus iridoid Ill, or a derivative thereof,

In one embodiment, the iridoid is loganin, or a derivative thereof.

In one embodiment, the iridoid is harpagoside, or a derivative thereof.

### Vector Combinations

As described herein, different combinations of enzymes present in the iridoid pathway and/or transcription factors result in increased expression of different iridoids.

There now follows non-limiting examples of different compositions designed to promote the expression of one or more iridoids.

In one embodiment, there is provided a composition for promoting the expression of one or more iridoids in a cell, wherein the composition comprises:
a) a vector component encoding MYB12.

In a further embodiment, the composition comprising a vector component encoding MYB12 promotes the expression of loganin and herpagoside.

In one embodiment, there is provided a composition for promoting the expression of one or more iridoids in a cell, wherein the composition comprises:
a) a vector component encoding DEL and ROS1.

In a further embodiment, the composition comprising a vector component encoding DEL and ROS1 promotes the expression of loganin and herpagoside.

In one embodiment, there is provided a composition for promoting the expression of one or more iridoids in a cell, wherein the composition comprises:
a) a vector component encoding MYB12, DEL, and ROS1.

In a further embodiment, the composition comprising a vector component encoding MYB12, DEL, and ROS1 promotes the expression of loganin.

In one embodiment, there is provided a composition for promoting the expression of one or more iridoids in a cell, wherein the composition comprises:
a) a vector component encoding TT8.

In a further embodiment, the composition comprising a vector component encoding TT8 promotes the expression of loganin.

In one embodiment, there is provided a composition for promoting the expression of one or more iridoids in a cell, wherein the composition comprises:
a) a vector component encoding AN2.

In a further embodiment, the composition comprising a vector component encoding AN2 promotes the expression of loganin and herpagoside.

In one embodiment, there is provided a composition for promoting the expression of one or more iridoids in a cell, wherein the composition comprises:
a) a vector component encoding MYB12 and TT8.

In a further embodiment, the composition comprising a vector component encoding MYB12 and TT8 promotes the expression of loganin and herpagoside.

In one embodiment, there is provided a composition for promoting the expression of one or more iridoids in a cell, wherein the composition comprises:
a) a vector component encoding MYB12 and AN2.

In a further embodiment, the composition comprising a vector component encoding MYB12 and AN2 promotes the expression of loganin.

In one embodiment, there is provided a composition for promoting the expression of one or more iridoids in a cell, wherein the composition comprises:
a) a vector component encoding TT8 and AN2.

In a further embodiment, the composition comprising a vector component encoding TT8 and AN2 promotes the expression of loganin.

In one embodiment, there is provided a composition for promoting the expression of one or more iridoids in a cell, wherein the composition comprises:
a) a vector component encoding MYB12, AN2, and TT8.

In a further embodiment, the composition comprising a vector component encoding MYB12, AN2, and TT8 promotes the expression of loganin and herpagoside.

In one embodiment, there is provided a composition for promoting the expression of one or more iridoids in a cell, wherein the composition comprises:
a) a vector component encoding DEL, ROS1, KOMT1, and SPS2.

In a further embodiment, the composition comprising a vector component encoding DEL, ROS1, KOMT1, and SPS2 promotes the expression of loganin.

In one embodiment, there is provided a composition for promoting the expression of one or more iridoids in a cell, wherein the composition comprises:
a) a vector component encoding MYB12, KOMT1, and SPS1.

In a further embodiment, the composition comprising a vector component encoding MYB12, KOMT1, and SPS1 promotes the expression of loganin.

In one embodiment, there is provided a composition for promoting the expression of one or more iridoids in a cell, wherein the composition comprises:
a) a vector component encoding DEL, ROS1, KOMT1, and SPS1.

In a further embodiment, the composition comprising a vector component encoding DEL, ROS1, KOMT1, and SPS1 promotes the expression of loganin.

In one embodiment, there is provided a composition for promoting the expression of one or more iridoids in a cell, wherein the composition comprises:
a) one or more vector components encoding one or more of DXS, GPPS, GES, G10H, GOR, ISY, MLPL, 10, 7-DLGT, 7-DLH and LAMT.

For example, the vector component may encode, one, two, three, four, etc or all of the listed enzymes and inserted into one or multiple vectors. For the expression of loganin, all of the listed enzymes must be transfected into the recipient plant cell.

In a further embodiment, the composition comprising a vector component encoding DXS, GPPS, GES, G10H, GOR, ISY, MLPL, 10, 7-DLGT, 7-DLH and LAMT promotes the expression of loganin.

The expressed iridoids, may or may not be endogenously present in the cell, cell line, yeast, bacteria, algae, moss, or plant in which the composition of the invention is used. It will be understood by the skilled person that where a particular iridoid is not endogenously produced, there is considered to be an increase in this iridoid if it can be detected after the cell, cell line, yeast, bacteria, algae, moss or plant has been transfected with the composition of the invention.

In situations where particular iridoids are endogenously expressed in the cell, cell line, yeast, bacteria, algae, moss, or plant before the composition of the invention is used, a 1-fold, 2-fold, 3-fold, 4-fold, 5-fold, 10-fold, 20-fold, 40-fold, 60-fold, 80-fold, 100-fold, 150-fold, 200-fold, 300-fold, 400-fold, 600-fold, 800-fold, 1000-fold, or more increase in that particular iridoid may be observed.

The total amount of iridoids present in a cell, cell line, yeast, bacteria, algae, moss, or plant in which the composition of the invention is used may be increased by 1-fold, 2-fold, 3-fold, 4-fold, 5-fold, 10-fold, 20-fold, 40-fold, 60-fold, 80-fold, 100-fold, 150-fold, 200-fold, 250-fold, or more.

The various aspects and/or embodiments described herein are presented only to assist in understanding and teaching the claimed features. These aspects and/or embodiments are provided as a representative sample of aspects and/or embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

The invention will now be described with reference to the following non-limiting examples.

### EXAMPLES

### Example 1: Promoting the Expression of Loganin and Harpagoside in Nicotiana tabacum with Transcription Factors Present in the Flavonoid Pathway

Plasmids comprising combinations of TT8, DEL, ROS1, AN2, and/or MYB12 were generated and used to transfect Nicotiana tabacum. The combinations were as set out in table 2 below, which corresponds to the results set out in table 3.

TABLE 2
Combinations of transcription factors in the plasmids of sets 1-9

| Set | Transcription Factors |
|---|---|
| 1 | MYB12 |
| 2 | DEL, ROS1 |
| 3 | MYB12, DEL, ROS1 |
| 4 | TT8 |
| 5 | AN2 |
| 6 | TT8, MYB12 |
| 7 | MYB12, AN2 |
| 8 | TT8, AN2 |
| 9 | TT8, MYB12, AN2 |
| 10 | control |

An Agrobacterium culture (Agrobacterium tumefaciens) was used to deliver the genetic material into the Nicotiana tabacum cells. The plasmids containing the genes of interest were introduced into separate Agrobacterium cultures and cultured overnight to reach specific density.

Once the specific density was reached (OD600 of ~1-2), it was resuspended at a density of OD600 0.2 and prepared as an infiltration solution. Infiltration solutions were prepared for each of the plasmid types (see table 2).

The infiltration solutions were then individually infiltrated into the leaves of the Nicotiana tabacum plants, using a syringe or vacuum infiltration. Separate plants were used for each set of transcription factors.

Following infiltration, the plants were incubated under controlled conditions (22° C., ~250 μmol/m2/s (light), 70% humidity and 16 hr day/night cycle.) in a growth chamber for 7 days. Following this, the infiltrated leaves were removed from the plant and frozen instantly.

The dried leaves were then ground and extraction was preformed using methanol. Extracts were then filtered and placed in vials to be analysed by HPTLC. Stationary Phase, Merck, HPTLC Silica gel 60 F₂₅₄, Mobile phase, Chloroform:Methanol 9:1 (v/v), Chloroform:Methanol:Water 80:20:2 (v/v/v), Chloroform:Methanol:Water 61:32:5 (v/v/v), Toluene:Ethyl acetate:Acetic acid:Chloroform 5:5:1:2 (v/v/v/v). After separation, the plates were derivatized with 10% H2SO4 in methanol and then visualized by normal light or UV lamp.

TABLE 3
fold change in iridoids after transfection
with the plasmids according to table 2.

| | fold change [set/control] | | | | | | | | |
|---|---|---|---|---|---|---|---|---|---|
| Com- | Set | | | | | | | | |
| pound | 1 | 2 | 3 | 4 | 5 | 6 | 7 | 8 | 9 |
| Harpa- | 1.54 | 1.45 | 0.92 | 0.91 | 1.81 | 1.20 | NA | 0.72 | 1.51 |
| goside | | | | | | | | | |
| Loganin | 1.23 | 1.18 | 1.69 | 1.39 | 1.91 | 1.91 | 1.69 | 1.80 | 2.04 |

The different transcription factor combinations, listed in table 2, had variable effects on the expression of loganin and harpagoside. As can be seen in table 3, the expression of loganin was increased (values 1) by all combinations of transcription factors. Some combinations were also able to increase the expression of harpagoside.

The increased expression of the iridoids in table 3 was a surprising finding, as it was expected that the transcription factors used would drive the generation of anthocyanins or flavonols instead of iridoids, as they are transcription factors associated with the flavonoid biosynthesis pathway. Furthermore, the results demonstrate that transcription factors alone are enough to increase the expression of iridoids.

### Example 2: Promoting the Expression of Loganin and Harpagoside in Nicotiana benthamiana with Enzymes and Transcription Factors Present in the Flavonoid Pathway

Plasmids comprising combinations of KOMT1, SPS1, SPS2, DEL, ROS1, and/or MYB12 were generated and used to transfect Nicotiana benthamiana. The combinations were as set out in table 4 below, which corresponds to the results set out in table 5.

TABLE 4
Combinations of enzymes and transcription
factors in the plasmids of sets 1-7

| Set | Enzymes/Transcription Factors |
|---|---|
| 1 | MYB12, KOMT1, SPS2 |
| 2 | DEL, ROS1, KOMT1, SPS2 |
| 3 | MYB12, DEL, ROS1, KOMT1, SPS2 |
| 4 | MYB12, KOMT1, SPS1 |
| 5 | DEL, ROS1, KOMT1, SPS1 |
| 6 | MYB12, DEL, ROS1, KOMT1, SPS1 |
| 7 | control |

An Agrobacterium culture (Agrobacterium tumefaciens) was used to deliver the genetic material into the Nicotiana benthamiana cells. The plasmids containing the genes of interest were introduced into separate Agrobacterium cultures and cultured to a specific density.

Once the specific density was reached (OD600 of ~1-2), it was resuspended at a density of OD600 0.2 and prepared as an infiltration solution. Infiltration solutions were prepared for each of the plasmid types (see table 4).

The infiltration solutions were then individually infiltrated into the leaves of the Nicotiana benthamiana plants, using a syringe or vacuum infiltration. Separate plants were used for each set.

Following infiltration, the plants were incubated under controlled conditions (22° C., ~250 μmol/m2/s (light), 70% humidity and 16 hr day/night cycle.) in a growth chamber for 7 days. Following this, the infiltrated leaves were frozen instantly.

The dried leaves were then ground and extraction was preformed using methanol. Extracts were then filtered and placed in vials to be analysed by HPTLC. Stationary Phase, Merck, HPTLC Silica gel 60 F₂₅₄, Mobile phase, Chloroform:Methanol 9:1 (v/v), Chloroform:Methanol:Water 80:20:2 (v/v/v), Chloroform:Methanol:Water 61:32:5 (v/v/v), Toluene:Ethyl acetate:Acetic acid:Chloroform 5:5:1:2 (v/v/v/v). After separation, the plates were derivatized with 10% H2SO4 in methanol and then visualized by normal light or UV lamp.

TABLE 5
fold change in iridoids after transfection
with the plasmids according to table 4.

| | fold change [set/control] | | | | | |
|---|---|---|---|---|---|---|
| | Set | | | | | |
| Compound | 1 | 2 | 3 | 4 | 5 | 6 |
| Harpagoside | NA | NA | NA | NA | NA | NA |
| Loganin | 0.97811 | 1.192721 | NA | 1.260146 | 1.061479 | NA |

As can be seen in table 5, three of the 6 combinations of enzymes and transcription factors from the flavonoid pathway were able to increase the expression of loganin (sets 2, 4 and 5) in Nicotiana benthamiana. However, sets 3 and 6 resulted in the loss of expression of loganin, which was found to be endogenously produced in Nicotiana benthamiana.

Harpagoside is not endogenously produced by Nicotiana benthamiana, nor was its expression able to be induced by the combinations of enzymes and transcription factors tried. Thus demonstrating that whilst enzymes and transcription factors from other biosynthesis pathways can be used to increase the expression of iridoids, careful selection is required to ensure that the desired terpene is generated.

### Example 3: Promoting the Expression of Loganin Nicotiana benthamiana with Loganin Genes

Plasmids individually encoding DXS, GPPS, GES, G10H, GOR, ISY, MLPL, 10, 7-DLGT, 7-DLH or LAMT were generated and used to transfect Nicotiana benthamiana.

An Agrobacterium culture (Agrobacterium tumefaciens) was used to deliver the genetic material into the Nicotiana benthamiana cells. The plasmids containing the genes of interest were introduced into separate Agrobacterium cultures and cultured overnight to reach specific density.

Once the specific density was reached (OD600 of ~1-2), it was resuspended at a density of OD600 0.2 and infiltration solutions were prepared.

The infiltration solutions were then infiltrated into the leaves of the Nicotiana benthamiana plants, using a syringe or vacuum infiltration, ensuring that the leaves had received each of the plasmid types and thus a combination of DXS, GPPS, GES, G10H, GOR, ISY, MLPL, IO, 7-DLGT, 7-DLH and LAMT.

Following infiltration, the plants were incubated under controlled conditions (22° C., ~250 μmol/m2/s (light), 70% humidity and 16 hr day/night cycle.) in a growth chamber for 7 days. Following this, the infiltrated leaves were removed from the plant and frozen instantly.

The dried leaves were then ground and extraction was preformed using methanol. Extracts were then filtered and placed in vials to be analysed by HPTLC. Stationary Phase; Merck, HPTLC Silica gel 60 F₂₅₄, Mobile phase; Chloroform-Ethanol-Methanol-0.5 M NaOH 30:15:2:1.5 (v/v). After separation, the plates were derivatized with 10% H2SO4 in methanol and then visualized by normal light or UV lamp.

Loganin is not endogenously expressed in Nicotiana benthamiana (see FIG. 10), thus clearly demonstrating that transfection of the plants with the plasmid encoding the loganin genes, results in expression of loganin.

1. A composition for promoting the expression of one or more iridoids in a cell, wherein the composition comprises:
a) one or more vector components encoding one or more enzymes present in the iridoid pathway; and/or
b) one or more vector components encoding one or more transcription factors.

2. The composition according to claim 1, wherein the one or more vector components encoding one or more enzymes present in the iridoid pathway are provided in a first vector and the one or more vector components encoding one or more transcription factors are provided in a second vector.

3. The composition according to claim 1, wherein the one or more vector components encoding one or more enzymes present in the iridoid pathway and one or more vector components encoding one or more transcription factors are provided in a single vector.

4. The composition according to any one or claims 1-3, wherein the one or more iridoids are one or more plant-derived iridoids.

5. The composition according to any one of claims 1-4, wherein the one or more transcription factors comprise myeloblastosis (MYB) type transcription factors and/or basic helix-loop-helix (bHLH) type transcription factors.

6. The composition according to any one of claims 1-5, wherein the one or more transcription factors are one or more transcription factors present in the iridoid pathway, and/or terpenoid pathway, and/or flavonoid pathway.

7. The composition according to any one or claims 1-6, wherein the one or more transcription factors comprise one or more of MYB12, AN2, ROS1, DEL, TT8, TSAR1, TSAR2, TSAR3, iridoid synthesis 1, iridoid synthesis 2, and iridoid synthesis 3.

8. The composition according any one of claims 1-7, wherein the one or more transcription factors are independently derived from a plant species.

9. The composition according to claim 8, wherein the plant species is selected from the group comprising At (Arabidopsis thaliana), Am (Antirrhinum majus), Nt (Nicotiana tabacum), Mt (Medicago truncatula), Vv (Vitis vinifera), or Cs (Camellia sinensis).

10. A plant, plant part or plant cell transformed with the vector components according to the composition of any one of claims 1-9.

11. The plant, plant part or plant cell according to claim 10, wherein the plant, plant part or plant cell is algae, moss or any plant, plant part or plant cell derived from the genus Nicotiana.

12. The plant, plant part or plant cell according to claim 11, wherein the plant, plant part or plant cell derived from the genus Nicotiana is selected from the group comprising Nicotiana tabacum, Nicotiana benthamiana, or BY-2 cells.

13. Use of the composition according to any one of claims 1-9 for promoting the expression of one or more iridoids in algae, moss, or any plant, plant part, or plant cells.

14. Use of the composition according claim 13, wherein the plant cells are a cell line derived from plants.

15. Use of the composition according to claim 13 or claim 14, wherein the plant, plant part, or plant cells are derived from the genus Nicotiana.

16. Use of the vector according to claim 15, wherein the plant, plant part, or plant cells derived from the genus Nicotiana are selected from the group comprising Nicotiana tabacum, Nicotiana benthamiana, or BY-2 cells.

17. Use of the vector according to any one of claims 13-16, wherein the one or more iridoids comprise one or more plant-derived iridoids.

18. A method of promoting the expression of one or more iridoids in a cell, comprising the step of:
a) introducing the composition according to any one of claims 1-9 into a cell.

19. The method according to claim 18, wherein the method further comprises:
b) extracting the one or more iridoids from the cell to produce an extract;
c) detecting the one or more iridoids in the extract;
d) purifying the one or more iridoids from the extract.

20. The method according to claim 19, wherein the one or more iridoids are purified using Flash and preparative MPLC, HPTLC or LC/MS.

21. The method according to any one of claims 18-20, wherein the cell is a plant cell.

22. The method according to claim 21, wherein the plant cell is an algae cell, moss cell, or any cell or cell line derived from the genus Nicotiana.

23. The method according to claim 22, wherein the cell or cell line derived from the genus Nicotiana is selected from the group comprising Nicotiana tabacum, Nicotiana benthamiana, or BY-2 cells.

24. The method according to any one of claims 18-23, wherein the one or more iridoids comprise one or more plant-derived iridoids.

25. An iridoid obtainable from the method according to any one of claims 18-24.

26. The iridoid according to claim 25, wherein the iridoid is a plant-derived iridoid.
